(19) United States
(12) Patent Application Publication
(10) Pub. No.: US 2005/0260219 A1
(43) Pub. Date: Nov. 24, 2005

(54) Carboxypeptidases B from anopheles gambiae. compositions comprising them, vaccine applications and use as therapeutical targets

(75) Inventors: Catherine Lavazec, New York, NY (US); Catherine Bourgouin, Saint Cyr - L'Ecole (FR); Christian Boudin, Juvignac (FR); Sarah Bonnet, Nantes (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris (FR)

(21) Appl. No.: 11/110,851

(22) Filed: Apr. 21, 2005

Related U.S. Application Data
(60) Provisional application No. 60/564,240, filed on Apr. 22, 2004.

(51) Int. Cl. C12Q 1/68; C07H 21/04; A61K 39/00; C12N 9/72

(52) U.S. Cl. 424/185.1; 435/6; 435/215; 435/348; 435/320.1; 536/23.2

(57) ABSTRACT

The present invention provides two carboxypeptidase B enzymes from Anopheles gambiae and homologs thereof. The present invention also provides compositions and vaccines containing the carboxypeptidase B enzymes, as well as antibodies directed thereto and various methods of using the same. The methods of the present invention include a method of blocking Plasmodium development and a method of identifying compounds that inhibit carboxypeptidase B activity.

### CROSS-REFERENCE TO RELATED APPLICATIONS

The present application claims priority to U.S. Application No. 60/564,240 filed on Apr. 22, 2004, which is hereby incorporated by reference in its entirety.

### BACKGROUND OF THE INVENTION

1. Field of the Invention

The present invention provides two carboxypeptidase B enzymes from Anopheles gambiae and homologs thereof. The present invention also provides compositions and vaccines containing the carboxypeptidase B enzymes, as well as antibodies directed thereto and various methods of using the same. The methods of the present invention include a method of blocking Plasmodium development and a method of identifying compounds that inhibit carboxypeptidase B activity.

2. Discussion of the Background

Anopheles gambiae is the main vector of Plasmodium falciparum, the most deadly species of human malaria parasites, and the most prevalent in Africa.

Upon ingestion by a female mosquito during a blood meal, Plasmodium gametocytes differentiate into male and female gametes, which fuse to form zygotes. Each zygote becomes a motile ookinete in the blood bolus and crosses the peritrophic matrix and the midgut epithelium to reach the hemocoel side of the gut. The ookinete attaches to the midgut wall and transforms into an oocyst, which undergoes numerous divisions to form sporozoites. Sporozoites are released into the hemolymph and invade salivary glands, where they attain maturity and can be injected into a new host during the next blood meal. During the transformation from gamete to oocyst that takes place inside the mosquito midgut, there is a considerable reduction in the number of parasites reaching the oocyst stage (6, 7).

Mosquito factors play a role in this parasite loss. Indeed, synthesis of nitric oxide (NO) was found to limit Plasmodium development in Anopheles stephensi (8). In addition, recent studies have demonstrated that An. gambiae genes involved in immunity pathways were activated during the sporogonic development of Plasmodium (9-12), suggesting that immune molecules might contribute to parasite loss. Furthermore, Osta and collaborators demonstrated that a gene encoding a leucine rich immune molecule controlled the number of parasites reaching the oocyst stage (13). Mosquito digestive enzymes could also affect the efficiency of parasite development. For instance, increased aminopeptidase activity has been reported in a strain of An. stephensi refractory to the development of P. falciparum (14).

Conversely, mosquito factors may facilitate the sporogonic development of Plasmodium. For example, mosquito xanthurenic acid has been identified as a gametocyte-activating factor, contributing to the maturation of male gametes (15, 16) and two C-type lectins were shown to protect Plasmodium ookinetes from melanization (13). Mosquito trypsin could affect the viability of ookinetes in vitro and possibly in vitro (17, 18), and it has also been proposed that this digestive enzyme could enhance Plasmodium infection by activating parasite chitinase, thereby facilitating ookinete passage through the peritrophic matrix, which surrounds the blood meal (19, 20). It has also been hypothesized that mosquito digestive proteases contribute to the successful development of Plasmodium by inactivating host complement and macrophages, to which parasites are sensitive (21, 22).

The spread of malaria continues despite intensive chemotherapeutic intervention and vector control campaigns. Several strategies are being developed to try and limit the global impact of malaria. The control of malaria is hampered by drug-resistance of malaria parasites and insecticide-resistance in mosquitoes. Understanding the intimate relationship between the parasite and the mosquito could provide new insights into rational methods for controlling malaria transmission (1-5).

Transmission of Plasmodium only occurs when sexual stages of parasites are ingested by a Anopheles female mosquito upon blood feeding on a gametocyte-carrying vertebrate and undergo sporogonic development within the mosquito vector. Therefore, one strategy to achieve malaria control is the development of transmission-blocking vaccines (TBVs), which will promote, in the vertebrate host, the production of antibodies inhibiting the parasite development in the mosquito midgut. Plasmodium transmission-blocking strategies have mainly targeted Plasmodium antigenic molecules expressed at the sexual and sporogonic stages of the parasite. Antibodies against these parasite molecules block parasite fertilization or prevent the passage of ookinetes across the mosquito midgut epithelium (1). For example, Pfs25, a molecule present on the surface of zygotes and ookinetes of the human malaria parasite Plasmodium falciparum, has been extensively investigated as a blocking immunogen (35-43).

An alternative approach consists in targeting mosquito molecules that the parasite encounters and relies on during sporogonic development. Antibodies directed against mosquito midgut homogenates or mosquito midgut glycoproteins were shown to inhibit Plasmodium sporogonic development (2, 5, 44-47). These data demonstrate that it is indeed feasible to limit Plasmodium development and transmission by antibodies targeted towards mosquito midgut determinants. However, the development of a TBV requires identification of a specific target. Whereas antibodies that inhibit trypsin activity in Aedes aegypti block transmission of the avian malaria parasite Plasmodium gallinaceum (48), no antigenic molecules have been identified as a component for a TBV in Anopheles mosquitoes, the vectors of the human malaria parasites.

Despite the aforementioned theories for the development Plasmodium infection and the heretofore developed strategies to treat and/or eradicate the same there remains a critical need for the development of viable safe methods to diminish the global impact of malaria.

### SUMMARY OF THE INVENTION

It is an object of the present invention to provide an isolated polynucleotide which encodes a polypeptide comprising an amino acid sequence that is at least 70% homologous to SED ID NO: 2 or SEQ ID NO: 4, wherein said polypeptide has carboxypeptidase B activity.

Another object of the present invention is to provide an isolated polynucleotide that has a sequence that is at least 70% homologous to nucleotides 32-1300 of SEQ ID NO: 1 or nucleotides 76-1341 of SEQ ID NO: 3.

Another object of the present invention is to provide a polynucleotide that is complementary to a polynucleotide which encodes a polypeptide comprising an amino acid sequence that is at least 70% homologous to SED ID NO: 2 or SEQ ID NO: 4.

Yet another object of the present invention is to provide a polynucleotide that hybridizes under stringent conditions to an isolated polynucleotide which encodes a polypeptide comprising an amino acid sequence that is at least 70% homologous to SED ID NO: 2 or SEQ ID NO: 4, wherein said polynucleotide encodes a polypeptide having carboxypeptidase B activity.

Another object of the present invention is to provide a vector comprising an isolated polynucleotide which encodes a polypeptide comprising an amino acid sequence that is at least 70% homologous to SED ID NO: 2 or SEQ ID NO: 4. In an embodiment of this object, the vector is operably linked to an inducible promoter.

It is still another object of the present invention to provide a host cell comprising the aforementioned vector, as well as a method for making a polypeptide that is at least 70% homologous to SEQ ID NO: 2 or SEQ ID NO: 4 and exhibits carboxypeptidase B activity, comprising culturing the host cell for a time and under conditions suitable for expression of said polypeptide, and collecting the said polypeptide.

Another object of the present invention is to provide a process for screening for a polynucleotide that encodes a protein having carboxypeptidase B comprising hybridizing a polynucleotide that is complementary to a polynucleotide which encodes a polypeptide comprising an amino acid sequence that is at least 70% homologous to SED ID NO: 2 or SEQ ID NO: 4 to the polynucleotide to be screened; expressing the polynucleotide to produce a protein; and detecting the presence or absence of carboxypeptidase B activity in said protein.

Still another object of the present invention is to provide a method for detecting a polynucleotide having at least 70% homology to an isolated polynucleotide which encodes a polypeptide comprising an amino acid sequence that is at least 70% homologous to SED ID NO: 2 or SEQ ID NO: 4 by contacting a nucleic acid sample with a probe or primer comprising at least 15 consecutive nucleotides of the aforementioned isolated polynucleotide, or at least 15 consecutive nucleotides of the complement thereof.

Another object of the present invention is to provide a method for producing a polynucleotide having at least 70% homology to an isolated polynucleotide which encodes a polypeptide comprising an amino acid sequence that is at least 70% homologous to SED ID NO: 2 or SEQ ID NO: 4 by:
(a) hybridizing a nucleic acid sample with a primer pair, wherein said primer pair comprises a first primer which is complementary to at least 15 consecutive nucleotides near the 5′end of the isolated polynucleotide, and a second primer which is complementary to at least 15 consecutive nucleotides near the 3′end of the isolated polynucleotide, and
(b) synthesizing said polynucleotide.

It is an object of the present invention to provide an isolated polypeptide comprising an amino acid sequence, which is at least 70% homologous to SEQ ID NO: 2 or 4 and exhibits carboxypeptidase B activity.

It is also an object of the present invention to provide an antibody directed against a polypeptide comprising an amino acid sequence, which is at least 70% homologous to SEQ ID NO: 2 or 4, or an immunogenic fragment thereof.

Another object of the present invention is to provide a method of blocking Plasmodium development (i.e., immunizing a subject) by administering to a subject (e.g., a mammal, preferably a human) in need thereof an effective amount of: (a) an antibody (e.g., a polyclonal or monoclonal antibody) directed against a polypeptide comprising an amino acid sequence, which is at least 70% homologous to SEQ ID NO: 2 or 4, or an immunogenic fragment thereof, or (b) a polypeptide comprising an amino acid sequence, which is at least 70% homologous to SEQ ID NO: 2 or 4, or an immunogenic fragment thereof.

It is also an object of the present invention to provide a vaccine comprising a polypeptide having an amino acid sequence, which is at least 70% homologous to SEQ ID NO: 2 or 4, or an immunogenic fragment thereof and at least one pharmaceutically acceptable carrier, adjuvant, diluent, or excipient.

Still another object of the present invention is to provide a method of identifying compounds that inhibit carboxypeptidase B activity by:
contacting a polypeptide comprising an amino acid sequence, which is at least 70% homologous to SEQ ID NO: 2 or 4, or a conformational epitope thereof, that possesses carboxypeptidase B activity with a carboxypeptidase B substrate selected from the group consisting of hyppuryl-arginine and hyppuryl-lysine in the presence of a candidate compound, and
measuring the residual carboxypeptidase B activity in the presence of said candidate compound compared to the carboxypeptidase B activity in the absence of said candidate compound.

In each of the objects above, wherein the polypeptide is 70% homologous to SEQ ID NO: 2, it is preferred that said polypeptide is at least 70% homologous to amino acid residues 20-423 of SEQ ID NO: 2, more preferably said polypeptide is at least 70% homologous to amino acid residues 115-423 of SEQ ID NO: 2.

In each of the objects above, wherein the polypeptide is 70% homologous to SEQ ID NO: 4, it is preferred that said polypeptide is at least 70% homologous to amino acid residues 20-422 of SEQ ID NO: 4, more preferably said polypeptide is at least 70% homologous to amino acid residues 108-422 of SEQ ID NO: 4.

The above objects highlight certain aspects of the invention. Additional objects, aspects and embodiments of the invention are found in the following detailed description of the invention.

### BRIEF DESCRIPTION OF THE FIGURES

The patent or application file contains at least one drawing executed in color. Copies of this patent or patent application publication with color drawing(s) will be provided by the office upon request and payment of the necessary fee.

A more complete appreciation of the invention and many of the attendant advantages thereof will be readily obtained as the same becomes better understood by reference to the following Figures in conjunction with the detailed description below.

FIG. 1—Alignment of CPBAg1 and CPBAg2 amino acid sequences.

Sequence comparison was performed using the Align.ppc program of MacMolly. Conserved residues are shaded. Arrows show predicted signal peptide cleavage, ● shows the presumed zymogen activation site for CPBAg1. Asterisks denote the residues involved in zinc and substrate binding. Aspartic acid at position 367 (boxed) is presumably involved in cleavage preference at arginine or lysines residue. Percentage identity between the two sequences is 32.3%. CPBAg1 (SEQ ID NO: 2) EMBL accession number: AJ627286; CPBAg2 (SEQ ID NO: 4) Ensembl accession number: ENSANGT00000020553.

FIG. 2—Quantitative expression of cpbAg1 (A) and cpbAg2 (B) in midgut or carcass of An. gambiae adults.

RNA was extracted from midguts or carcasses of sugar-fed adult mosquitoes, reversed transcribed and amplified by real-time PCR. All data were normalized to the expression level level of the ribosomal protein S7 gene. The normalized expression levels were plotted as % of the normalized expression in female midguts set at 100. Bars indicate standard deviation from at least five PCR experiments.

FIG. 3—Blood-meal effect on cpbAg1 (A) and cpbAg2 (B) expression in female midguts.

Real-time PCR expression of cpbAg1 (A) and cpbAg2 (B) at different time points post emergence and post blood-meal in midguts of An. gambiae females. Data were normalized to the expression level level of the ribosomal protein S7 gene. The normalized expression levels were plotted as % of the normalized expression in midguts of 2-day old females set at 100. Unfed d2 and unfed d5: midguts from unfed mosquitoes 2 days and 5 days after emergence, respectively; BM: blood-meal; PBM: post blood-meal. Bars indicate standard deviation from at least five PCR experiments.

FIG. 4—Quantitative expression of cpbAg1 (A, B, C) and cpbAg2 (D, E, F) in An. gambiae midguts after ingestion of Plasmodium falciparum.

Real-time PCR expression of cpbAg1 (A, B, C) and cpbAg2 (D, E, F) at different time points (14 h, 24 h, 48 l h) after ingestion of gametocyte-containing blood (grey bars) or asexual stage containing blood (white bars). Results were plotted as % of the expression level in midguts of mosquitoes fed on non-infected control blood set at 100% (black bars), after normalization of the data to the expression level of the ribosomal protein S7 gene. Bars indicate standard deviation from at least five PCR experiments. * Significant, P<0.05.

FIG. 5—Quantitative expression of cpbAg1 (A, B, C) and cpbAg2 (D, E, F) in An. gambiae midguts after ingestion of Plasmodium berghei.

Real-time PCR expression ratio of cpbAg1 (A, B, C) and cpbAg2 (D, E, F) at different time points (14 h, 24 h, 48 h) after feeding on P. berghei 2.34 infected mice (gametocyte-producing producing strain, grey bars) or on P. berghei 2.33 infected mice (non-gametocyte-producing strain, white bars). Results were plotted as % of the expression level in midguts of mosquitoes fed on noninfected mice, set at 100 (black bars) after normalization of the data to the expression level of the ribosomal protein S7 gene. Bars indicate standard deviation from at least five PCR experiments. * Significant, P<0.05.

FIG. 6—Specificity and effect on CPB activity of the anti-CPBAg1 serum. (FIG. 6A): Analysis of the anti-CPBAg1 serum specificity by western-blot. Lane 1: 0.1 ng of CPBAg1 recombinant protein; lane 2: 1 μg of protein from blood-fed mosquito midgut (24 h PBM); lane 3: 20 ng of CPBAg2 recombinant protein. The blot was probed either with anti-CPBAg1 rabbit serum or naive rabbit serum at a dilution of 1:500. The anti-CPBAg1 serum, unlike the non-immune rabbit serum (not shown), recognizes two bands corresponding to the uncleaved (48.2 KDa) and cleaved (37 KDa) CPBAg1 and CPBAg2 proteins in mosquito midguts.

(FIG. 6B): Effect of the anti-CPBAg1 serum on CPB activity in mosquito midguts. CPB activity was determined in midguts isolated at 14 h, 24 h, and 48 h after ingestion of uninfected human red blood cells mixed with anti-CPBAg1 serum or with naive rabbit serum as a control. The data represent % of inhibition of CPB activity in mosquitoes fed on anti-CPBAg1 serum compared to that detected in mosquitoes fed on naive rabbit serum. The Inhibition was calculated as %=100×[1-(CPB activity in anti-CPBAg1 serum fed mosquitoes/CPB activity in control mosquitoes)], using the mean value from each series of three experiments. PBM: time post blood meal.

FIG. 7—Carboxypeptidase activity in non-infected and P.falciparum infected An. gambiae midgut. CPB activity was determined in midguts isolated before and at 14 h, 24 h, and 48 h after ingestion of a non-infected human blood meal (●) and P. falciparum infected human blood meal (◯). One unit of enzyme activity is defined as μmol of amino acids released/min. Bars indicate standard deviation from three independent experiments.

FIG. 8—Immunization protocol for groups of mice.

Mice from group 1 and group 2 were immunized with the recombinant CPBAg1 protein on day 0. Mice from group 1 (8 immunized and 8 control mice) were inoculated with P. berghei on day 21 and were used for mosquito feeding and bled for ELISA experiments (*) on day 24. Mice from group 2 (3 immunized and 3 control mice) were boosted on day 21, were inoculated with P. berghei on day 42 and were used for mosquito feeding and bled for ELISA experiments (*) on day 45. *: 100 μl blood sample per mouse were collected for ELISA experiments after mosquito feeding.

FIG. 9—Comparison of CPBAg1 specific ELISA titers in the sera from immunized and control mice.

Specific antibody titers against CPBAg1 were measured in duplicates for each mice by ELISA three weeks after the first (group 1) and the booster (group 2) immunization. The average reading at O.D. 490 nm of each group was plotted against the reciprocal sera dilution. End points were defined as the highest serial dilutions yielding an absorbance reading at 490 nm greater than 0.5. (Δ) and (□) show antibody titers from group 1 and 2 respectively in sera from immunized mice. (▴) and (▪) show antibody titers from group 1 and 2 respectively in sera from control mice.

FIG. 10—Analysis of antibody recognition of mosquito midgut proteins.

M: 10 μg of protein from sugar-fed mosquito midgut, CPBAg1: 10 ng of CPBAg1 recombinant protein. The blot was probed either with sera from CPBAg1 immunized mice or pool of sera from control mice at a dilution of 1:200. The sera from CPBAg1 immunized mice, unlike the pool of sera from control mice (not shown), specifically recognize two bands corresponding to the uncleaved (48.2 KDa) and cleaved (37 KDa) CPBAg1 protein in mosquito midguts.

FIG. 11—Effect of antibodies on parasite development.

Intensity of infection (number of parasites per positive midgut) by P. berghei was scored by detection of GFP-expressing parasites in midguts of mosquitoes fed on control (black bars) and immunized mice (grey bars). Gametocytemia was determined just before feeding by Giemsa staining of thin smears and by GFP fluorescence detection. Mosquitoes were dissected at 3 h, 24 h, 48 h and 9 days after blood feeding to visualize zygotes (Zyg), ookinetes (Ook), young oocysts (Ooc 48 h) and mature oocysts (Ooc day 9), respectively. Geometric mean intensity for six immunized mice was plotted as % of that for six control mice set at 100%. The table indicates the geometric mean number of parasite per midgut at each parasite life stages.

FIG. 12—Alignment of CPBAg1 (SEQ ID NO: 2) with six carboxypeptidase sequences.

Sequence comparison was performed using ClustalW multiple sequence alignment. Conserved residues are shaded in grey, and residues involved in zinc and substrate binding are boxed. id, sim: percentages of identity (id) and similarity (sim) between CPBAg1 and each of carboxypeptidase sequences. The NCBI accession numbers for each of the sequences are as follows: CPB1 human (NP_{—}001862; SEQ ID NO: 61), CPB2 human (AAT97987; SEQ ID NO: 62), CPB porcine (P09955; SEQ ID NO: 63), CPB rat (NP_{—}036665; SEQ ID NO: 64), CPA Anopheles (AAB96576; SEQ ID NO: 65) and CPA Aedes (AAD47827; SEQ ID NO: 66).

FIG. 13—Comparison of the three-dimensional structural model of CPBAg1 and three-dimensional structure of porcine carboxypeptidase B.

Modelization of the three-dimensional structure of CPBAg1 (panels B and D) as obtained with ProModII program and compared to porcine pro-carboxypeptidase B (panels A and C; PDB access code: 1nsa). (A and B) Overall structure of porcine CPB and CPBAg1 as a ribbon plot, with α-helices represented as ribbons) and β-strands as arrows. The pro-domain structure of both CPBs is coloured in orange and the active-enzyme moiety in blue. The zinc ion is shown as a green sphere. (C and D) Comparison of active site and substrate binding pocket of porcine CPB and CPBAg1, respectively. The side chains of important amino acids are shown as a stick model. Zinc ion (in green) is coordinated by the side chains of three amino acids highlighted in red. Side chains of amino acids that are involved in the interaction with the substrate are colored in yellow. The Asp residue that confers specificity of the enzyme to the basic penultimate amino acid of the substrate is indicated in green.

FIG. 14—Alignment of the conserved sequences involved in substrate specificity and activity in predicted zinc-carboxypeptidases of Anopheles gambiae.

The twenty-two putative carboxypeptidases deduced from the An. gambiae genome were aligned with CPBAg1 using ClustalW. Only the regions of sequence involved in substrate specificity and activity are shown. Z, bold: zinc binding; C, dark grey background: catalytic activity; S, grey background: substrate binding; and S*, grey background: substrate specificity of the enzyme with a D at this position for CPBs or a hydrophobic amino acid (L, I, V or P) for CP As. cp-like4 and cp-like7 sequences likely correspond to truncated or wrongly predicted genes.

FIG. 15—Phylogenetic tree based on sequence similarity among Anopheles gambiae zinc-carboxypeptidases.

Sequence alignment of all predicted zinc-carboxypeptidases (family M14, clan MC) from the An. gambiae genome were aligned using ClustalW and a phylogenetic tree was then generated using Phylip parsimony analysis. The names of the different genes are as described in Table 3, which contains the NCBI accession numbers. Identical colour codes highlight clustering of the corresponding genes on chromosomes (CHR).

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, bioinformatics, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention relates to two genes from Anopheles gambiae encoding carboxypeptidase B proteins.

As used herein, the term “cpbAg1” is used to designate the polynucleotide sequence of SEQ ID NO: 1. Specifically, the term “cpbAg1” is used to designate the open-reading frame appearing at nucleotides 32-1300 of SEQ ID NO: 1. In the same manner, the term “cpbAg2” is used to designate the polynucleotide sequence of SEQ ID NO: 3. Specifically, the term “cpbAg2” is used to designate the open-reading frame appearing at nucleotides 76-1341 of SEQ ID NO: 3.

As used herein, the term “CPBAg1” is used to designate the polynucleotide sequence of SEQ ID NO: 2 (i.e., the full-length zymogen—amino acid residues 1-423 of SEQ ID NO: 2), the pro-protein (amino acid residues 20-423 of SEQ ID NO: 2, and/or the mature protein (amino acid residues 115-423 of SEQ ID NO: 2). In the same manner, the term “CPBAg2” is used to designate the polynucleotide sequence of SEQ ID NO: 4 (i.e., the full-length zymogen—amino acid residues 1-422 of SEQ ID NO: 4), the pro-protein (amino acid residues 20-422 of SEQ ID NO: 4, and/or the mature protein (amino acid residues 108-422 of SEQ ID NO: 4).

The present inventors first identified cDNA from Anopheles gambiae corresponding to a gene hereafter named cpbAg1. The deduced amino acid sequence harbors the features of caboxypeptidase proteins of the B type. The cpbAg1 cDNA harbors similarity to another predicted transcript from An.gambiae (hereafter named cpbAg2), which is another embodiment of the present invention.

Both transcripts for CPBAG1 and CPBAG2 are expressed in the An. gambiae female midgut at a high level and their expression is increased in mosquitoes that have ingested P. falciparum gametocytes.

Therefore, in one object of the present invention are the isolated polynucleotide sequences cpbAg1 and cpbAg2, homologs thereof (at least 70%, preferably at least 80%, more preferably at least 90%, and most preferably at least 95%), vectors containing the same, and host microorganisms that have been transformed with said vector (e.g., bacterial, viral, mammalian, insect, plant, etc.).

In another object of the present invention, the vector containing the DNA of the present invention (and/or DNA encoding the inventive protein sequences) serves to provide enhanced protein production and/or activity. The term “enhanced” as used herein means increasing the intracellular activity or concentration of the CPAG1 and CPAG2 proteins, which are encoded by the corresponding DNA. Enhancement can be achieved with the aid of various manipulations of the bacterial cell. In order to achieve enhancement, particularly over-expression, the number of copies of the corresponding gene can be increased, a strong promoter can be used, or the promoter- and regulation region or the ribosome binding site which is situated upstream of the structural gene can be mutated. Expression cassettes that are incorporated upstream of the structural gene act in the same manner. In addition, it is possible to increase expression by employing inducible promoters. A gene can also be used which encodes a corresponding enzyme with a high activity. Expression can also be improved by measures for extending the life of the mRNA. Furthermore, preventing the degradation of the enzyme increases enzyme activity as a whole. Moreover, these measures can optionally be combined in any desired manner. These and other methods for altering gene activity in a plant are known as described, for example, in Methods in Plant Molecular Biology, Maliga et al, Eds., Cold Spring Harbor Laboratory Press, New York (1995).

Within the present invention, it is contemplated that the gene (or DNA) may be a part of a fusion construct to facilitate purification. Examples of such fusion proteins encoded thereby and the corresponding purification methods are well appreciated by the skilled artisan and need not be described here. These examples include GST-tags, His-tags, FLAG-tags, etc. Preferred examples include GST-tags and His-tags.

The present invention also includes polynucleotides that hybridize to the polynucleotide sequences of cpbAg1 and cpbAg2, or homologs and/or fragments thereof, under stringent conditions.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 5×SSC, preferably 1× to 2×SSC, (20×SSC=3.0 M NaCl/0.3 M trisodium citrate) at 50 to 68° C., preferably 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem., 138:267-284 (1984): Tm=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

In the context of the present invention, a polynucleotide sequence is “homologous” with the sequence according to the invention if at least 70%, preferably at least 80%, more preferably at least 90%, most preferably at least 95% of its base composition and base sequence corresponds to the sequence according to the invention (i.e., cpbAg1 and cpbAg2).

In another object of the present invention are the polypeptide sequences corresponding to CPBAG1 and CPBAG2, or a homolog thereof. The polypeptides of the present invention exhibit carboxypeptidase B activity.

The present invention also provides a method of obtaining a protein having carboxypeptidase B activity by expressing a polynucleotide encoding a protein having the sequence of CPBAG1 and CPBAG2, or a homolog thereof, for a time, under conditions, and in a medium suitable for said expression, and subsequently recovering (and/or isolating or purifying) the resultant protein.

Fermentation or culturing is in general carried out at a pH of 5.5 to 9.0, in particular 6.0 to 8.0. Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum or the desired level of CPBAG1 or CPBAG2, or a homolog thereof, has formed. This target is usually reached within 10 hours to 160 hours. The appropriate growth media would be selected for the corresponding host microorganism, the identity of which would be readily apparent to the skilled artisan.

According to the invention, a “homologous protein” or “homologous polypeptide” is to be understood to comprise proteins (polypeptides) which contain an amino acid sequence at least 70% of which, preferably at least 80% of which, more preferably at least 90%, most preferably at least 95% of which corresponds to the amino acid sequence of CPBAG1 and CPBAG2. It is particularly preferred that the homologous protein retain at least 50%, preferably at least 70%, more preferably at least 80%, most preferably at least 90% of the residual activity of the wild-type CPBAG1 and CPBAG2 proteins. It is particularly preferred that the homologous polypeptide retain at least 50%, preferably at least 70%, more preferably at least 80%, even more preferably 90%, most preferably at least 95% of the residual immunogenicity of the corresponding immunogenic fragment of the CPBAG1 and CPBAG2 proteins.

The expression “homologous amino acids” denotes those that have corresponding properties, particularly with regard to their charge, hydrophobic character, steric properties, etc.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Sequence alignments can also be performed using the Align.ppc program (Mac Molly TetraLite, Mologen) or ClustalW.

One skilled in the art is also aware of conservative amino acid replacements such as the replacement of glycine by alanine or of aspartic acid by glutamic acid in proteins as “sense mutations” which do not result in any fundamental change in the activity of the protein, i.e. which are functionally neutral. It is also known that changes at the N— and/or C-terminus of a protein do not substantially impair the function thereof, and may even stabilize said function.

Polynucleotide sequences according to the invention are also suitable as primers for polymerase chain reaction (PCR) for the production of DNA, which encodes a CPBAG1 or CPBAG2 protein.

Oligonucleotides that may serve as probes or primers can contain at least 30, more preferably at least 20, most preferably at least 15 consecutive nucleotides of SEQ ID No: 1, preferably of nucleotides 32-1300 of SEQ ID NO: 1, or SEQ ID NO: 3, preferably of nucleotides 76-1341 of SEQ ID NO: 3. Further, the polynucleotides that may serve as probes or primers can be complementary to at least 30, more preferably at least 20, most preferably at least 15 consecutive nucleotides of SEQ ID No: 1, preferably of nucleotides 32-1300 of SEQ ID NO: 1, or SEQ ID NO: 3, preferably of nucleotides 76-1341 of SEQ ID NO: 3. Oligonucleotides with a length of at least 40 or 50 nucleotides are also suitable.

Further, the probes or primers of the present invention may contain at least 30, more preferably at least 20, most preferably at least 15 consecutive nucleotides of a polynucleotide sequence that encodes a polypeptide that is at least 70% (preferably 80%, more preferably at least 90%, most preferably at least 95%) homologous to SEQ ID No: 2 or SEQ ID NO: 4. Even further, the polynucleotides that may serve as probes or primers can be complementary to at least 30, more preferably at least 20, most preferably at least 15 consecutive nucleotides of consecutive nucleotides of a polynucleotide sequence that encodes a polypeptide that is at least 70% (preferably 80%, more preferably at least 90%, most preferably at least 95%) homologous to SEQ ID No: 2 or SEQ ID NO: 4.

The term “isolated” means separated from its natural environment.

The term “polynucleotide” refers in general to polyribonucleotides and polydeoxyribonucleotides, and can denote an unmodified RNA or DNA or a modified RNA or DNA. Further, this term embraces recombinant polynucleotides. Of course, this term also embraces salt forms thereof.

The term “polypeptides” is to be understood to mean peptides or proteins that contain two or more amino acids that are bound via peptide bonds. Further, this term embraces recombinant polypeptides. Of course, this term also embraces salt forms thereof.

The present inventors have produced a recombinant protein corresponding to a fusion of CPBAG1 and GST in E. coli. However, the present invention also provides recombinant proteins that exist in the absence of tags and/or fusion constructs. The inventors have used said recombinant GST-CPBAG1 fusion to produce an immune rabbit serum. This serum exhibits a blocking effect on the development of P. falciparum when added to an infective blood meal containing P. falciparum gametocytes.

Also, a recombinant protein corresponding to a fusion of CPBAG1 to HIS-Tags was produced in the baculovirus system. The purified recombinant His-Tag protein was used to immunize mice. When An. gambiae female mosquitoes feed on such mice immunized and infected with the rodent parasite P. berghei, the development of the parasite in the mosquito midgut is considerably reduced compared to mosquitoes fed on non-immunized but infected mice, using the same procedures.

These results evince that CPBAG1 protein shows features of a target antigen for P. falciparum transmission blocking vaccine. Considering the phylogenetic links among Anopheles mosquitoes, the present invention may be extended to other Anopheles vectors of P. falciparum and Anopheles vectors of Plasmodium sp.

Decreasing or inactivating carboxypeptidase B activity within the midgut bolus of An. gambiae female mosquitoes with specific inhibitors, defined on conformational epitopes of CPBAG1 and CPBAG2 or gathered from high throughput technology, would led to the blocking of P. falciparum development. Such inhibitors would constitute therapeutic complement to control the transmission of P. falciparum from mosquitoes to humans (or from humans to mosquitoes).

Accordingly, it is an object of the present invention to provide vaccines based on the newly identified carboxypeptidase B proteins (CPBAG1 and CPBAG2) As used herein, the term “vaccine” is not limited to just an immunogenic composition able to induce an individual protection against malaria. As used herein, the term “vaccine” is used in the context of providing protection against malaria by the way of community protection by blocking parasite transmission among their hosts (vertebrate-insect and vice-versa). The latter definition is different from vaccines designed to block or kill the parasite itself. Advantageously, the transmission-blocking proffered by the present invention by abolishing carboxypeptidases activities is independent of the parasite development stage. To this end, the experimental work conducted by the inventors reveals that mammals are able to develop antibodies that can block the transmission of plasmodium parasites, from the mosquitoes that transported them, when they are immunized with said carboxypeptidase B antigens.

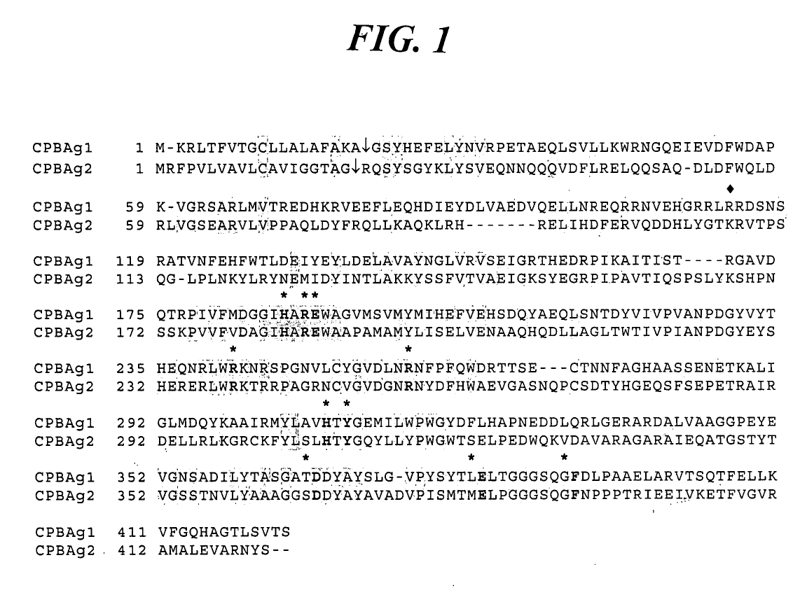

Therefore, the present invention provides immunogenic fragments of CPBAG1 and CPBAG2. In an embodiment of the present invention, the immunogenic fragments correspond to regions having homology between CPBAG1 and CPBAG2 (see, for example, FIGS. 1 and 14 for regions having high homology). In another embodiment of the present invention, the immunogenic fragments are non-linear-conformational epitopes.

The term “immunogenic fragment” as it relates to polypeptides is understood to mean a polypeptide fragment of at least 6 amino acids and preferably at least 10 amino acids sufficient to induce an immune response when it is administered to a host eukaryotic organism.

The polypeptides and/or immunogenic fragments thereof of the present invention are administered in a dose, which is effective to vaccinate (i.e., elicit an immunogenic response) a mammal, preferably a human, against malaria infection or to treat a mammal, preferably a human, having a malaria infection. As used herein, an effective amount of the polypeptides to achieve this goal is generally from about 2 ng to 2 mg/kg of body weight per week, preferably about 2 μg/kg per week. This range includes all specific values and subranges therebetween.

Examples of other animals (i.e., host eukaryotic organisms) envisioned within the purview of the present invention include: a rodent animal, a camel (camelid animal), a monkey (non-human primate).

It is to be understood that the polypeptides and/or immunogenic fragments thereof may be administered as a pharmaceutical composition containing the polypeptide compound and a pharmaceutically acceptable carrier, diluent, excipient, or adjuvant. The active materials can also be mixed with other active materials, which do not impair the desired action and/or supplement the desired action. Any route can be used to administer the active materials according to the present invention, for example, orally, parenterally, intravenously, intradermally, subcutaneously, or topically, in liquid or solid form.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

Another mode of administration of the polypeptides of this invention is oral. To this end, oral administration is envisaged by the present inventors to entail, in addition to conventional modes (see below), transformation of plants used as a host feed a vector or genomic integration of a nucleotide sequences coding for the entire or partial antigens of the present invention (i.e., creating transgenic plants). Among appropriate transgenic plants for use in this embodiment of the present invention are any plant used for food consumption by a host organism, for example corn, tomatoes, etc. Tomatoes, however, are particularly preferred.

Therefore, as used herein the term “host cells” is used to embrace animal cells, as well as plant cells.

Oral compositions will generally include an inert diluent or an edible carrier. For the purpose of oral therapeutic administration, the aforesaid polypeptides may be incorporated with excipients and used in the form of tablets, gelatine capsules, troches, capsules, elixirs, suspensions, syrups, wafers, chewing gums and the like. Compositions may be prepared according to any method known to the art for the manufacture of pharmaceutical compositions and such compositions may contain one or more agents selected from the group consisting of sweetening agents, flavoring agents, coloring agents and preserving agents. Tablets containing the active ingredient in admixture with nontoxic pharmaceutically acceptable excipients, which are suitable for manufacture of tablets, are acceptable. These excipients may be, for example, inert diluents, such as calcium carbonate, sodium carbonate, lactose, calcium phosphate or sodium phosphate granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques to delay disintegration and adsorption in the gastrointestinal tract and thereby provide a sustained action over a longer period. For example, a time delay material such as glyceryl monostearate or glyceryl distearate alone or with a wax may be employed. Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

The tablets, pills, capsules, troches and the like may contain the following ingredients: a binder such as microcrystalline cellulose, gum tragacanth or gelatin; an excipient such as starch or lactose, a disintegrating agent such as alginic acid, Primogel, corn starch and the like; a lubricant such as magnesium stearate or Sterotes; a glidant such as colloidal silicon dioxide; and a sweetening agent such as sucrose or saccharin or flavoring agent such as peppermint, methyl salicylate, or orange flavoring may be added. When the dosage unit form is a capsule, it may contain, in addition to material of the above type, a liquid carrier such as a fatty oil. Other dosage unit forms may contain other various materials, which modify the physical form of the dosage unit, for example, as coatings. Thus tablets or pills may be coated with sugar, shellac, or other enteric coating agents. A syrup may contain, in addition to the active polypeptides, sucrose as a sweetening agent and certain preservatives, dyes and colorings and flavors. Materials used in preparing these various compositions should be pharmaceutically or veterinarially pure and non-toxic in the amounts used.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropylethyl cellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension may also contain one or more preservatives such as ethyl or n-propyl p-hydroxybenzoate, one or more coloring agents, one or more flavoring agents and one or more sweetening agents, such as sucrose, aspartame, saccharin, or sucralose.

Oil suspensions may be formulated by suspending the active ingredient in a vegetable oil, such as arachis oil, olive oil, sesame oil or coconut oil, or in a mineral oil such as liquid paraffin. The oil suspensions may contain a thickening agent, such as beeswax, hard paraffin or cetyl alcohol. Sweetening agents may be added to provide a palatable oral preparation. These compositions may be preserved by the addition of an antioxidant such as ascorbic acid.

Dispersible powders and granules of the invention suitable for preparation of an aqueous suspension by the addition of water may be formulated from the active ingredients in admixture with a dispersing, suspending and/or wetting agent, and one or more preservatives. Suitable dispersing or wetting agents and suspending agents are exemplified by those disclosed above. Additional excipients, for example sweetening, flavoring and coloring agents, may also be present.

The compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The compositions of the invention may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a nontoxic parenterally acceptable diluent or solvent, such as a solution of 1,3-butanediol. Among the acceptable vehicles and solvents that may be employed are water and Ringer's solution, an isotonic sodium chloride. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Sterilization may be performed by conventional methods known to those of ordinary skill in the art such as by aseptic filtration, irradiation or terminal sterilization (e.g. autoclaving).

Aqueous formulations (i.e oil-in-water emulsions, syrups, elixers and injectable preparations) may be formulated to achieve the pH of optimum stability. The determination of the optimum pH may be performed by conventional methods known to those of ordinary skill in the art. Suitable buffers may also be used to maintain the pH of the formulation.

The polypeptides of this invention may also be administered in the form of suppositories for rectal administration of the drug. These compositions can be prepared by mixing the drug with a suitable nonirritating excipient, which is solid at ordinary temperatures but liquid at the rectal temperatures and will therefore melt in the rectum to release the drug. Non-limiting examples of such materials are cocoa butter and polyethylene glycols.

Methods for the production of antibodies directed to a specific peptide or fragment thereof are well known by those of skill in the art. Antibodies can be obtained by injecting an animal with an immunogenic polypeptide of the present invention, or an immunogenic fragment thereof, and recovering the antibodies which are able to complex with said immunogenic peptide or fragment thereof from said animal. Examples of such methods are disclosed in Antibodies, A Laboratory Manual, Harlow and Lane, Cold Spring Harbor Press, 1988, herein incorporated by reference.

Further, the antibodies of the present invention may be either polyclonal antibodies or monoclonal antibodies directed against either CPBAG1 or CPBAG2. For creation of polyclonal antibodies, rabbits and mice are mention as preferable mammals to raise the same. Techniques for making monoclonal antibodies by hybridoma are well known to the skilled artisan.

Techniques that make it possible to humanize antibodies have been described in the following references: Waldmann T. (1991). Science. 252: 1657-1662; Winter G. et al. (1993). Immunology Today. 14(6): 243-246; Carter et al. (1992). Proc. Natl. Acad. Sci, USA. 89: 4285-4289; and Singer et al. (1993). Journal of Immunology. 150(7): 2844-2857, each of which are incorporated herein by reference.

In another embodiment of the present invention is a process for screening for a polynucleotide that encodes a protein having carboxypeptidase B comprising hybridizing a polynucleotide that is complementary to a polynucleotide which encodes a polypeptide comprising an amino acid sequence that is at least 70% (preferably 80%, more preferably 90%, most preferably 95%) homologous to CPBAG1 or CPBAG2 to the polynucleotide to be screened; expressing the polynucleotide to produce a protein; and detecting the presence or absence of carboxypeptidase B activity in said protein.

In still another embodiment of the present invention is a method for detecting a polynucleotide having at least 70% homology to an isolated polynucleotide which encodes a polypeptide comprising an amino acid sequence that is at least 70% (preferably 80%, more preferably 90%, most preferably 95%) homologous to CPBAG1 or CPBAG2 by contacting a nucleic acid sample with a probe or primer comprising at least 15 consecutive nucleotides of the aforementioned isolated polynucleotide, or at least 15 consecutive nucleotides of the complement thereof.

Another embodiment of the present invention is a method for producing a polynucleotide having at least 70% homology to an isolated polynucleotide, which encodes a polypeptide comprising an amino acid sequence that is at least 70% (preferably 80%, more preferably 90%, most preferably 95%) homologous to CPBAG1 or CPBAG2 by:
(a) hybridizing a nucleic acid sample with a primer pair, wherein said primer pair comprises a first primer which is complementary to at least 15 consecutive nucleotides near the 5′ end of the isolated polynucleotide, and a second primer which is complementary to at least 15 consecutive nucleotides near the 3′ end of the isolated polynucleotide, and
(b) synthesizing said polynucleotide.

Another embodiment of the present invention is a method of blocking Plasmodium development (i.e., immunizing a subject) by administering to a subject (e.g., a mammal, preferably a human) in need thereof an effective amount of: (a) an antibody (e.g., a polyclonal or monoclonal antibody) directed against a polypeptide comprising an amino acid sequence, which is at least 70% (preferably 80%, more preferably 90%, most preferably 95%) homologous to CPBAG1 or CPBAG2, or an immunogenic fragment thereof, or (b) a polypeptide comprising an amino acid sequence, which is at least 70% (preferably 80%, more preferably 90%, most preferably 95%) homologous to CPBAG1 or CPBAG2, or an immunogenic fragment thereof.

Still another object of the present invention is to provide a method of identifying compounds that inhibit carboxypeptidase B activity by:
contacting a polypeptide comprising an amino acid sequence, which is at least 70% (preferably 85%, more preferably 90%, most preferably 95%) homologous to CPBAG1 or CPBAG2, or a conformational epitope thereof, that possesses carboxypeptidase B activity with a carboxypeptidase B substrate selected from the group consisting of hyppuryl-arginine and hyppuryl-lysine in the presence of a candidate compound, and
measuring the residual carboxypeptidase B activity in the presence of said candidate compound compared to the carboxypeptidase B activity in the absence of said candidate compound.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Material and Methods Common to All Examples

Mosquitoes

All experiments were performed with Anopheles gambiae Yaoundé strain (24) either at the Pasteur Institute (Paris, France) or at the IRD (Yaoundé, Cameroon and Dakar, Senegal). Mosquitoes were reared at 26° C. and 80% relative humidity, under a 12 h light/dark cycle. Dissections were performed in cold phosphate-buffered saline at 4° C. Midguts and carcasses (whole mosquito minus midgut) were stored at −80° C. until RNA or protein extraction.

### Example 1

### Identification and Cloning of cpbAg1 and cpbAg2

Materials and Methods

Mosquitoes

The mosquitoes used for this example were essentially as described above; however, mosquitoes were reared at 26 ° C. and 80% relative humidity, under a 12 h light/dark cycle. Experimental feedings on human blood were performed with nulliparous females (5 days old) starved from sugar for 24 h, using the artificial membrane feeding technique (24). Mosquitoes were fed for 10 min. Just before feeding the mosquitoes, red blood cells were centrifuged for 10 min at 800 g, washed three times in RPMI incomplete medium (Gibco-BRL, Gaithersburg, Md., USA), and resuspended in human AB serum. From the fully fed females, midguts were isolated and pools of forty midguts were made at 3, 6, 9, 12, 15, 18, 24 and 48 h post blood-meal (PBM). All dissections were performed in cold phosphate-buffered saline at 4 ° C. Midguts and carcasses (whole mosquito minus midgut) were stored at −80 ° C. until RNA or protein extraction.

cybAg1 Cloning, Production of a GST Recombinant Protein and Related Antibodies

A full length cDNA corresponding to cpbAg1 was obtained by 5′ RACE using the following specific primers derived from an amplicon previously selected by DDRT-PCR (26; Genebank accession number AF348131): GeneRacer 5′ (5′-CGACTGGAGCACGAGGACACTGA-3′; SEQ ID NO: 11), GeneRacer 5′nested (5′-GGACACTGACATGGACTGAAGGAGTA-3′; SEQ ID NO: 12), CPBAgRace (5′-AGCGCGATAGCGTAACTGCTAGAA-3′; SEQ ID NO: 13), and CPBAgnestedRace (5′-TTGCGACTGCTCAATAGCAACGACTT-3′; SEQ ID NO: 14). A 1.5 kb gel-purified PCR product was cloned into Topo-TA cloning vector pCR-40® (Invitrogen, Cergy-Pontoise, France) leading to plasmid pcpbAg1, and sequenced (ABI PRISM™ 310 Genetic Analyser, Applied Biosystems, Fostercity, Calif., USA). The Ensembl Mosquito Genome (Anopheles gambiae genome) and the NCBI (National Library of Medicine, National Institutes of Health website) servers were used for sequence similarity analysis and genome annotation. Analysis for potential signal peptide was carried out using the SignalP 3.0 Server Web site available through the Center for Biological Sequence Analysis—Technical University of Denmark DTU. Sequence alignments were performed using the Align.ppc program (Mac Molly TetraLite, Mologen) or ClustalW. Parsimony analysis (Phylip) was used to generate a phylogenetic tree edited with TreeView (below).

Also formed was a CPBAg1-GST fusion recombinant protein corresponding to CPBAg1 minus its signal sequence, fused to GST at its N-terminal end was produced in Escherichia coli strain BL21, using pGEX 3× expression vector (Amersham Pharmacia Biotech). The purified fusion protein was used to produce anti-CPBAg1 antibodies in rabbit (Biogenes).

Homology Modeling of CPBAg1

CPBAg1 three-dimensional structure was modeled according to structures of laye.pdb (Human Procarboxypeptidase A2), 1jqg.pdb (Helicoverpa armigera; carboxypeptidase A), 1pca.pdb (Porcine (Sus scrofa) pancreas; Procarboxypeptidase A), 1nsa.pdb (Sus scrofa; Procarboxypeptidase B), and 2ctb.pdb (Bovine (Bos taurus) pancreas; Carboxypeptidase A) carboxypeptidases. The ProModII program was used to model protein environment (58). Energy minimization was carried out with the GROMOS96 force field (Parameter file IFP43B 1; Topology file MTB43B 1; method one: steepest descent, 200 cycles, 25/C-factors constraints; conjugate gradient method, 300 cycles, 2500/C-factors constraints).

Production of a Recombinant GST fusion Protein in E. coli and Related Antibodies

To construct a recombinant GST fusion protein, a DNA fragment covering the cpbAg1 open reading frame without its signal peptide sequence was amplified by PCR from pcpbAg1 using the following primers containing restriction sites for BamHI and HindIII: CPBAg1express3′: 5′-GGCGAATTCCAGATCTCTAAGCTTCGAAGTCACCGACAGTGT (SEQ ID NO: 15); CPBAg1express5′: 5′-TAAAAGCTGAATTCCGGGATCCCTTCGAGCT GTACAACGTG (SEQ ID NO: 16). The PCR product was cloned into pGEX 3× (Amersham Pharmacia Biotech, Orsay, France) leading to pcpbAg1-GST. The production of the recombinant fusion protein was done in E. coli BL21 as follows: after induction with IPTG for 3 h at 37° C., bacteria were centrifuged and disrupted in STETGST buffer (10 mM Tris, 150 mM NaCl, 1 mM EDTA, 5 mM DTT, 1% lysozyme and 1.5% sarcosyl) by sonication. The GST fusion protein was then purified by affinity on glutathione-agarose beads (Sigma, Lyon, France). The purified protein was used to produce anti-CPBAg1 antibodies in rabbits (Biogenes, Berlin, Germany).

Production of a His-Tagged Recombinant Protein in a Baculovirus Expression System

Cells and virus culture. Spodoptera frugiperda (Sf9) and Trichoplusia ni (High Five, HF) insect cells (Invitrogen, Cergy-Pontoise, France) either in monolayer or in suspension cultures were cultured at 27° C. in SF-900 medium (Gibco-BRL, Gaithersburg, Md., USA) or insect X-Press medium (Biowhittaker, Baltimore, Md., USA) supplemented with 4 mM glutamine (Gibco-BRL, Gaithersburg, Md., USA), respectively. Amplification of recombinant viruses and virus stocks (10⁸ pfu/ml) was performed in Sf9 cells. Protein expression was carried out in HF cells.

Construction. The construct used to express the recombinant protein contained the whole CPBAg1 open reading frame fused to a His-Tag at its C-terminal end. First, a DNA fragment corresponding to the 423 amino acids of the protein CPBAg1 was generated by PCR using pcpbag1 as a template and the following primers: pVLcpbAg1-5′: 5′-TAACGGATCCGGATGAGCGAAGCAATG (SEQ ID NO: 17); pVLcpbAg1-3′: 5′-AATCTAGACTATTAGTGGTGGTGGTGGTGGTGCGAAGTCACCGACAGTGT (SEQ ID NO: 18). After digestion with BamHI and XbaI, the PCR fragment was cloned into the BamHI/XbaI cut transfer vector pVL1393 (Invitrogen, Cergy-Pontoise, France) leading to plasmid pVLcpbAg1 whose sequence was verified. Recombinant viruses were obtained by gently mixing 15 μg recombinant transfer vector with 50 ng BaculoGold viral DNA (PharMingen, SanDiego, Calif., USA) in 1.5 ml TC100 medium (Gibco-BRL, Gaithersburg, Md., USA) supplemented with 4 mM glutamine and 50 μg/ml gentamycine (Gibco-BRL, Gaithersburg, Md., USA) and then mixed with an equal amount of medium containing 50 μl DOTAP (Liposomal Transfection Reagent, Roche, Meylan, France). After a 10 min incubation step, this mix was then added to 2.5×10⁶ Sf9 cells in T-25 culture flasks and cultured for 6 days with one change of medium after 24 h. Recombinant viruses were then isolated from the culture supernatant by plaque assay using standard methods (59).

Expression and Purification of Recombinant His-Tag CPBAg1

Around 1.6×10⁶ HF cells/ml were infected with recombinant viruses (multiplicity of infection=10) and grown in suspension cultures, at 27° C. for 3 days, in X-Press medium supplemented with 50 μg/ml gentamycine. Purification was performed using an AKTA purifier system (Amersham Pharmacia Biotech, Orsay, France). Culture supernatants were centrifuged for 15 min at 1800×g to remove cells and cellular debris, dialysed against 20 mM Tris-HCl/300 mM NaCl, pH 8 and applied to a 1 ml HITRAP™ chelating HP column (Amersham Pharmacia Biotech, Orsay, France) loaded with CoCl₂ 0.1 M, equilibrated in the same buffer. The column was extensively washed and bound protein eluted with a linear gradient from 0 to 100% of 200 mM imidazole in 20 mM Tris-HCl (pH 8)/300 mM NaCl. CPBAg1 recombinant protein was eluted with 20 mM imidazole. One millilitre fractions were collected and analysed by electrophoresis and Western blotting. The purified CPBAg1 recombinant protein was subjected to amino-terminal Edman degradation carried out on an Applied Biosystems Sequencer at the Laboratoire de Microséquencage des Protéines, Institut Pasteur, Paris, France. Both forms of CPBAg1 were isolated from poly acrylamide gel and subjected to N-terminal sequence analysis.

Protein Extraction and Western Blot

Proteins were prepared from midguts and carcasses by tissue homogenization in 25 mM Tris-HCl pH 8 using a plastic pestle and further centrifugation at 20 000 g, 4° C., for 15 min. Soluble protein fractions and the His-tagged recombinant CPBAg1 protein were analysed on a 12% SDS-poly acrylamide gel under reducing conditions. Proteins were visualized by Coomassie staining (Biosafe®, Biorad, Marnes-la-Coquette, France) or electrophoretically transferred on to a PVDF membrane (Hybond-P, Amersham, Orsay, France) for Western blotting. After saturation in PBS containing 5% skimmed milk and 0.1% Tween 20, the membrane was incubated with purified anti-CPBAg1 antibodies (dilution 1:500). Antibody binding was detected by incubating the membrane with mouse anti-rabbit peroxidase-conjugated antibody (SantaCruz, dilution 1:10⁵) and subsequent treatment with a peroxidase chemiluminescent substrate (SuperSignal Ultra®, Pierce, Brebières, France)

Enzymatic Assays

Carboxypeptidase activity was determined using the synthetic di-peptide substrates Hippuryl-Arginine (Hip-Arg), Hippuryl-Lysine (Hip-Lys) as carboxypeptidase B substrates and Hippuryl-Phenylalanine (Hip-Phe) as carboxypeptidase A substrate. All reagents were purchased from Sigma. Activities were assayed in 20 μl E buffer (100 mM NaCl/50 mM Hepes/100 μM ZnCl₂ pH 7.2) containing 1 mM Hip-Arg, Hip-Lys or Hip-Phe. The reaction was initiated by adding an equal volume of His-tagged CPBAg1 (5 ng/μl in E buffer), incubated at 25° C. for 10-40 min, and stopped by the addition of 20 μl Ninhydrin reagent (Sigma, Lyon, France). The rate of the reaction was measured by estimating the amount of released amino acids using the ninhydrin procedure (60). One unit of enzyme activity was defined as μmol of amino acids released/min.

Estimation of Km and kcat was carried out after assays over a 500-fold range of Hip-Arg concentration (0.02-10 mM). Each concentration was assayed in triplicate. The kinetic parameters were derived from iterative non-linear least square fits of the Michaelis-Menten equation using the experimental data (61). Confidence limits for the fitted values were determined by 100 Monte Carlo iterations using the experimental standard deviations on individual measurements.

Inhibitory effects of the chelating agent 1,10-phenantroline (Sigma, Lyon, France), and of the arginine analogue GEMSA (Guanidinoethylmercaptosuccinic acid, Fluka) on CPBAg1 activity were assayed as follows: different concentrations (10⁴-1 mM) of inhibitors were preincubated at 25° C. for 30 min with the recombinant protein in E buffer before addition of Hip-Arg. Activity was measured as described above and was expressed as the percentage of activity relative to controls performed in absence of inhibitor.

RNA Extraction

Total RNA was extracted from each mosquito sample using the Tri Reagent® kit (M.R.C.Inc, Ontario, Canada) according to the manufacturer's instructions and RNA was treated with the DNA-free® kit (Ambion, Austin, Tex., USA). Absence of contaminating genomic DNA in each RNA sample was checked by specific amplification of the cpbAg1 gene using the following primers: cpbAg1U: 5′-GGCGGCTGAGGCGTGACT (SEQ ID NO: 5) and cpbAg1L: 5′-GACGGGTCTGATCGACTG (SEQ ID NO: 6).

Standard RT-PCR

RNA from two independent mosquito samples corresponding to the same experimental conditions were pooled and reverse-transcribed as follows: 100 ng pooled RNA was incubated with a random hexamer mixture and MMLV reverse transcriptase (400 units per reaction, Invitrogen) in a final volume of 40 μl, for 1 h at 42° C. followed by 5 min at 95° C. RT assays were performed in triplicate and RT products were pooled. PCR amplification was then performed in duplicate on 1 μl of pooled RT products. Amplification conditions were: five touchdown cycles (30 s at 94° C., 1 min at 5° C. above the Tm of the specific primers, 1 min at 72° C.) followed by thirty cycles (30 s at 94° C., 1 min at the Tm of the specific primers, 1 min at 72° C.) and a final 10 min elongation step (72° C.). Products were analysed on 1% agarose gels in Tris-borate buffer. The twenty-three specific pairs of primers corresponding to cpbAg1 and each of the twenty-two other predicted carboxypeptidase genes were: cpbAg1 (5′-GGCGGCTGAGGCGTGACT; SEQ ID NO: 5) and (5′-GACGGGTCTGATCGACTG; SEQ ID NO: 6); cpbAg2 (5′-TCCGGCACAATTGGACTACT; SEQ ID NO: 9) and (5′-TACCGCAGGTACTTGTTGAG; SEQ ID NO: 10); cpbAg3 (5′-GGGACTATCTGTGGGTGT; SEQ ID NO: 19) and (5′-GACCGCAATGTTCGATCCG; SEQ ID NO: 20); cpbAg4 (5′-ACGAAAGCAATCGGCAAGA; SEQ ID NO: 21) and (5′-TTGGACGTGCCCTGGCCGC; SEQ ID NO: 22); cpbAg5 (5′-GCCGGTTGTCTTCATCGACGGTGGT; SEQ ID NO: 23) and (5′-TTGGCAACCGGCACGATGACGTAGT; SEQ ID NO: 24); cpaAg1 5′-GGAGAAGCTGCGCGGTAAGCTCGGT; SEQ ID NO: 25) and (5′-ACTAAAAGCAGCGCTAAGCCACCAA; SEQ ID NO: 26); cpaAg2 (5′-CTACTACGCGACGATTGC; SEQ ID NO: 27) and (5′-GTTCGGGATGATCTGGTTA; SEQ ID NO: 28); cpaAg3 (5′-TATATCGAATCTCCAAGA; SEQ ID NO: 29) and (5′-GTGCCGTAACGATTGTAA; SEQ ID NO: 30); cpaAg4 (5′-GGTCGGGAAGGAAACGCTCGCGGCAT; SEQ ID NO: 31) and (5′-ACTCCACTCTCACGCACAGGGGGAAC; SEQ ID NO: 32); cpaAg5 (5′-TAAGTATCAGTGGATCGTT; SEQ ID NO: 33) and (5′-TATTGCGCGACTTTCTGGC; SEQ ID NO: 34); cp-like1 (5′-CTCTGGGCCATCGCCATT; SEQ ID NO: 35) and (5′-GGTCAGCGGAGTTTGAAGA; SEQ ID NO: 36); cp-like2 (5′-TTCCACTCTCACGTAAGC; SEQ ID NO: 37) and (5′-CACCCCGTACGGTTGCAT; SEQ ID NO: 38); cp-like3 (5′-ATCAATAGGCAGCAAGAGAAGAAAA; SEQ ID NO: 39) and (5′-GGATTTTTATCGTCGGGGTTTACCT; SEQ ID NO: 40); cp-like4 (5′-TTCATATTTCTAGACTATCCCACAC; SEQ ID NO: 41) and (5′-GCTCTGGACGGTAAGAATGGAGGA; SEQ ID NO: 42); cp-like5 (5′-CGCCGCACCAAAACACGCTACCTAA; SEQ ID NO: 43) and (5′-ACCTTCGTGTAGGTGTTGCGCTTCA; SEQ ID NO: 44); cp-like6 (5′-ACAACCGATAAGGCACCA; SEQ ID NO: 45) and (5′-CACTGCCATTGCTGTAAT; SEQ ID NO: 46); cp-like7 (5′-GGACTATAAGTGATTATC; SEQ ID NO: 47) and (5′-GTGCAAGATCCGATAGTG; SEQ ID NO: 48); cp-like8 (5′-TTCGCAAGCTGCGGCCTGCGTGCAAG; SEQ ID NO: 49) and (5′-GCGCCACCGGTACCTTCTCGATGTC; SEQ ID NO: 50); cp-like9 (5′-GTGGAACACCGTTGGTGGAAGCAA; SEQ ID NO: 51) and (5′-AACGCTGTTGGCAAGATTGCGCAG; SEQ ID NO: 52); cp-like10 (5′-CAAGCATTCGAAGAACTCAAACACA; SEQ ID NO: 53) and (5′-CTGGATTCGCTATCGGAATAATGAT; SEQ ID NO: 54); cp-like11 (5′-CGGCCAAACTTCGCCTACAATCAGA; SEQ ID NO: 55) and (5′-CCAAAGGTACGTATCTCGTATGACC; SEQ ID NO: 56); cp-like12 (5′-ATCGTATGAAGATACTCA; SEQ ID NO: 57) and (5′-TATACCCTTCCCGCAGGA; SEQ ID NO: 58); cp-like13 (5′-GGTAGAGGAGAACGTACAGTCACTGC; SEQ ID NO: 59) and (5′-ATCGATCGCATCCTCATCGACTTC; SEQ ID NO: 60).

Quantitative RT-PCR

Quantitative RT-PCR was performed to analyze in detail the expression pattern of cpbAg1. RNA pools were prepared as described above as pools of RT reactions. Real-time PCR was performed using the dsDNA dye SyberGreen® (MasterMix Perkin Elmer, Wellesley, Mass., USA) and the iCycler® apparatus from BioRad. PCR experiments were performed in quintuplet in a 25 μl final volume containing 900 nM of each forward and reverse primer and 5 μl of a ⅕ dilution of the RT product. Relative quantification of cpbAg1 mRNA was performed using the standard curve method (User Bulletin 2, ABI) and the ribosomal protein S7 mRNA as endogenous reference. The relative amount of cpbAg1 mRNA and S7 mRNA was determined from a standard curve constructed using an MRNA sample of known concentration. The primers used for the amplification of cpbAg1 were as described above and those for S7 amplification were: S7U: 5′-CACCGCCGTGTACGATGCCA-3′ (SEQ ID NO: 7); S7L: 5′-ATGGTGGTCTGCTGGTTCTT-3′ (SEQ ID NO: 8).

### RESULTS AND DISCUSSION

Identification of cpbAg1 a Novel Gene from An. gambiae Encoding a Carboxypeptidase B

Based on the previous differential display analysis aimed at identifying An. gambiae genes regulated by the P. falciparum parasite, the present inventors isolated the 3′ end of a cDNA corresponding to a transcript, named 55Yde, whose expression was down-regulated 14 h after ingestion of a gametocyte-containing blood meal (26). Using a 5′RACE strategy, the present inventors isolated the 5′ end of this cDNA and the assembled fill-length sequence of 1471 bp (GENBANK accession number AJ627286) harbors an open reading frame of 1269 bp coding for a protein of 423 residues, with a predicted molecular mass of 48.2 kDa. Consultation of the An. gambiae genome database indicated that cpbAg1 is localized on chromosome 2L and contains three introns. BLAST search revealed that the predicted amino acid sequence shares significant similarity with carboxypeptidases from various organisms. Indeed, it displayed between 48 and 54% similarity (28-32% identity) to carboxypeptidases from insect or mammalian species (FIG. 12) (54,56,57,62-64). This gene was thereafter named cpbAg1, as the first described gene encoding an An. gambiae carboxypeptidase B.

A blast search on the An. gambiae Ensembl server indicated that cpbAg1 (ENSANGT00000020640) belongs to a family of 23 carboxypeptidases, only five of which harbor features of active carboxypeptidases of the B class (not shown). RT-PCR experiments (not shown) indicated that only two of these putative carboxypeptidase B-encoding genes, cpbAg1 and ENSANGTOO000020553, named thereafter cpbAg2, were expressed at a significant level in An. gambiae midgut (see below). cpbAg1 cDNA shares 56.3% nucleotide identity to the predicted cpbAg2 cDNA and the corresponding proteins 32.3% identity. An alignment of CPBAG1 and CPBAG2 is shown in FIG. 1.

Carboxypeptidases belong to a family of zinc-containing exopeptidases that catalyse the hydrolysis of C-terminal amino acids, specifically lysines and arginines for carboxypeptidases B. In haematophagous insects, they are usually involved in blood digestion (21-23). As cpbAg1 and cpbAg2 were expressed in the midgut of adult mosquitoes, the present inventors investigated their expression pattern in mosquitoes fed on uninfected and Plasmodium infected blood.

Structural Features of CPBAg1

Sequence analysis of the predicted protein suggests that it is first translated as a pre-pro-protein, with a signal sequence between amino acids 1-19 and a pro-domain between amino acids 20 and 114. The presence of a putative signal sequence suggests that zymogen is secreted and the CPBAg1 zymogen activated after tryptic cleavage at Arg114 releasing the pro-domain. These features are consistent with other characterized carboxypeptidases involved in digestive processes that are also translated as pre-pro-proteins (54,55).

Using the three-dimensional structure of five carboxypeptidases (PDB access code: 1aye, 1jpg, 1pca, 1nsa and 2ctb), the present inventors constructed a three-dimensional model of CPBAg1. Comparison with porcine pro-protein carboxypeptidase B (1nsa; 62) indicated a similar global conformation fold (FIGS. 13A,B). The pro-domain (in orange), which interacts with the substrate binding pocket of the active-enzyme moiety displays a classically globular β₁α₁β₂β₃α₂β₄ topology. The active moiety of CPBAg1 (FIGS. 13A,B, in blue) displays a similar fold as the active moiety of porcine CPB, with eight strands of parallel/antiparallel βsheets surrounded by eight α-helices.

The active site of CPBAg1 is predicted to consist of His187, Glu190 and His308 which coordinate the zinc ion. As in the porcine CPB structure, residues Glu382 and Arg242 that are involved in the polarization and cleavage of scissible carbonyl group of the substrate are conserved and are located in proximity to the zinc ion.

The peptide-binding pocket is also conserved between both CPBs with the presence of Arg189, Tyr310, Phe391, Arg259, Tyr360 and Asp367 in the same spatial location for CPBAg1. This suggests that Tyr360 and Arg259 likely stabilize the terminal carboxylate group of the peptide substrate and Asp367 defines the specificity of the enzyme for basic C-terminal side chain residue (66). These characteristics classify CPBAg1 in the B class of carboxypeptidases.

Characterization of Recombinant cpbAg1

To determine whether cpbAg1 codes for an active carboxypeptidase B and to characterize the enzymatic activity, cpbAg1 was expressed in insect cells using the baculovirus expression system. Purification yielded two proteins with apparent molecular masses of 50 and 37 kDa, respectively and determination of their N-terminal amino acids demonstrated that the 50 kDa band corresponds to zymogen and the 37 kDa band to the mature form of CPBAg1 . Therefore, cleavage of the signal peptide occurs between Arg19 and Gly20, and that of the pro-peptide between Arg114 and Asp115.

Activity of the purified recombinant CPBAg1 produced in baculovirus was assayed against the carboxypeptidase B di-peptide substrates Hippuryl-Arginine (Hip-Arg) and Hippuryl-Lysine (Hip-Lys) and the carboxypeptidase A di-peptide substrate Hippuryl-Phenylalanine (Hip-Phe). 1 unit cleaves 1 μmol product/min. The results are shown in Table 1:

TABLE 1
Activity assay on recombinant CPBAg1.

| Substrates | Enzyme activity unit/mg |
|---|---|
| Hip-Arg | 0.83 ± 0.01 |
| Hip-Lys | 0.24 ± 0.02 |
| Hip-Phe | 0 |

As shown in Table 1, recombinant CPBAg1 exhibited activity against Hip-Arg and Hip-Lys, whereas no enzymatic activity could be quantified with the carboxypeptidase A substrate Hip-Phe. The estimated activity against Hip-Arg was approximately 3.5 times higher than with Hip-Lys suggesting that CBPAg1 has a preference for cleaving arginine residues.

Accordingly, CPBAg1 activity was inhibited by the active site-directed inhibitor GEMSA, which is an arginine analogue (Table 2). Activity of the purified recombinant CPBAg1 produced in the baculovirus was assayed after incubation with the zinc-carboxypeptidase inhibitor 1,10-phenantroline and the active site-directed inhibitor GEMSA, using Hyp-Arg as substrate. 100% activity corresponds to 1.1 and 0.8 unit/mg for 1,10-phenanatroline and GEMSA experiments, respectively.

TABLE 2
Effect of inhibitors on CPBAg1 activity.

| Inhibitor | Concentration (μM) | Activity (%) |
|---|---|---|
| 1,10-phenantroline | 0 | 100 ± 5 |
| | 0.1 | 96 ± 2 |
| | 10 | 87 ± 8 |
| | 100 | 35 ± 5 |
| | 1000 | 24 ± 5 |
| GEMSA | 0 | 100 ± 10 |
| | 0.1 | 96 ± 2 |
| | 10 | 41 ± 0 |
| | 100 | 20 ± 2 |
| | 1000 | 7 ± 11 |

In addition, the general zinc-protease inhibitor 1,10-phenantroline inhibited CPBAg1 activity, probably by chelating the zinc ion cofactor. These data confirmed that cpbAg1 encodes an active carboxypeptidase B, consistent with the database searches. The kinetic parameters for hydrolysis of Hip-Arg were then determined by the standard Michaelis-Menten method using varying substrate concentrations and the Kₘ was estimated as 2.59 mM with a specificity constant (k_{cat}/Kₘ) of 0.31×10⁵ M/S.

cybAg1 and cpbAg2 are Specifically Expressed in An. gambiae Midgut and Their Expression is Regulated Upon Blood Feeding

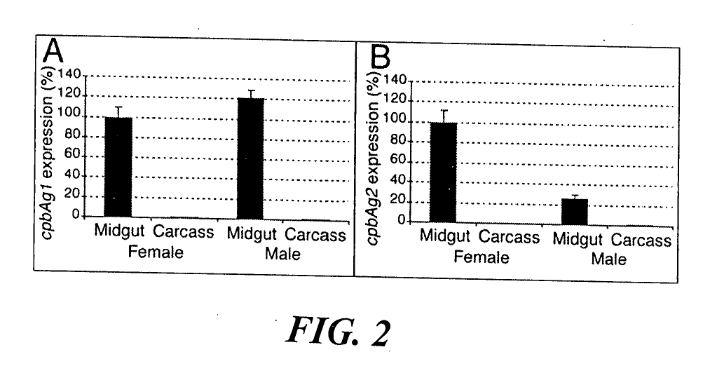

To determine the expression pattern of cpbAg1 and cpbAg2 in adult mosquitoes, real-time RT-PCR experiments were performed with RNAs extracted from midguts and carcasses of sugar-fed males and females. As shown in FIG. 2, in sugar-fed mosquitoes both cpbAg1 and cpbAg2 were mainly expressed in the midguts, while no expression was quantifiable in carcasses. Whereas the level of cpbAg1 expression was similar between male and female midguts, the level of cpbAg2 expression in males represented only 26% of its expression in females. The expression of cpbAg1 and cpbAg2 was comparable in midguts of sugar-fed females (ratio cpbAg1/cpbAg2: 1.3).

The midgut specific expression pattern of CPBAg1 was confirmed using specific antibodies directed against the recombinant enzyme. Consistently, CPBAg1 was found only in the midgut, with no signal detected in carcasses. As observed during purification of the recombinant protein, two endogenous polypeptides corresponding to the pro-enzyme and the processed enzyme were detected.

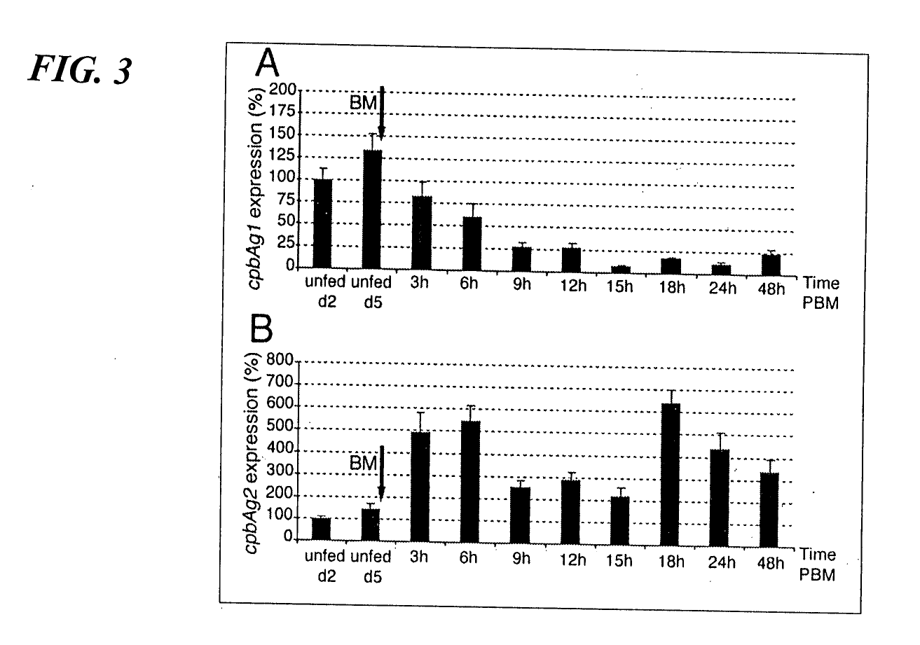

As both genes were expressed mainly in midguts, the present inventors investigated their expression in female midguts at various times before and after a non-infected blood-meal. As shown in FIG. 3A, cpbAg1 mRNA level in midguts of sugar-fed females increased slightly between day 2 and day 5 post emergence. On day 5, mosquitoes were fed on non-infected blood and the cpbAg1 MRNA level was found to decrease gradually over 48 hours. The lowest mRNA level, which was detected 15 h post blood-meal (PBM), was about 10 fold less than the level detected in midguts of sugar-fed females. At 96 h PBM, cpbAg1 expression returned to its initial level before the blood-meal. A subsequent blood-meal triggered the same pattern of gene expression (data not shown). By contrast, expression of cpbAg2 was increased after a bloodmeal in two waves: from 3 h to 6 h PBM and from 18 h to 24 h PBM (FIG. 3B). In between, although the amount of detected cpbAg2 decreased, it was still 2 to 3 times higher than in sugar-fed midguts.

cpbAg1 Belongs to a Family of Twenty-One Carboxypeptidase Genes

Interrogation of the An. gambiae genome database at Ensembl (release 11.2.1) indicated that cpbAg1 belongs to a family of twenty-one genes encoding twenty-three putative carboxypeptidases (as one annotated gene was predicted to give rise to three transcripts, including cpbAg1). Analysis of the twenty-three predicted proteins highlighted ten sequences containing residues known to be required for carboxypeptidase function. Five sequences, including cpbAg1, harbour hallmarks of CPB and were given a serial number (Table 3), whereas five other sequences displayed hallmarks of CPA (67,68) and were labeled cpaAg1 to cpaAg5. The gene characterized by Edwards et al. (56) and formerly named AgCP, corresponds to cpaAg1. The remaining thirteen sequences did not contain all of the carboxypeptidase finger-prints and were therefore labelled cp-like 1 to cp-like 13. An alignment of the homologous regions of the An. gambiae carboxypeptidase sequences is presented in FIG. 14.

TABLE 3
Characteristics and expression of predicted Anopheles gambiae CPs.

| | | | | | | | | | Gene expression | |
|---|---|---|---|---|---|---|---|---|---|---|
| | Predicted transcript | | | | Predicated protein | | | | Sugar fed | |
| Transcript | Accession | Chromosome | e | Length | Length | CP | Signal | Pro- | mosquitoes | |
| name | number | location | Value | (bp) | (aa) | prints | Csequence | peptide | Midgut | Carcass |

Predicted CPB

| | | | | | | | | | | |
|---|---|---|---|---|---|---|---|---|---|---|
| cpbAg1 | XM_316277 | 2 | 0.0 | 1287 | 423 | CPB | Yes | Yes | ++ | + |
| cpbAg2 | XM_316270 | 2 | 3E−68 | 1317 | 438 | CPB | Yes | Yes | ++ | + |
| cpbAg3 | XM_316274 | 2 | 4E−113 | 1248 | 415 | CPB | Yes | Yes | +/− | +/− |
| cpbAg4 | XM_310460 | X | 3E−59 | 1248 | 416 | CPB | No | Yes | − | − |
| cpbAg5 | XM_316275 | 2 | 5E−149 | 819 | 273 | CPB | No | No | − | − |

Predicted CPA

| | | | | | | | | | | |
|---|---|---|---|---|---|---|---|---|---|---|
| cpaAg1 | XM_318615 | 3 | 1E−57 | 1332 | 443 | CPA | Yes | Yes | ++ | − |
| cpaAg2 | XM_317081 | 3 | 5E−55 | 1272 | 423 | CPA | Yes | Yes | ++ | − |
| cpaAg3 | XM_317083 | 3 | 7E−62 | 1266 | 421 | CPA | Yes | Yes | ++ | ++ |
| cpaAg4 | XM_309221 | X | 7E−54 | 1368 | 455 | CPA | Yes | Yes | ++ | ++ |
| cpaAg5 | XM_317088 | 3 | 3E−47 | 867 | 289 | CPA | No^{a} | No^{a} | ++ | ++ |

Predicted CP-LIKE

| | | | | | | | | | | |
|---|---|---|---|---|---|---|---|---|---|---|
| cp-like1 | XM_317868 | 3 | 5E−52 | 1287 | 428 | NO | Yes | No | ++ | ++ |
| cp-like2 | XM_309222 | X | 4E−52 | 1359 | 452 | NO | Yes | Yes | ++ | ++ |
| cp-like3 | XM_317392 | 3 | 2E−42 | 1065 | 354 | NO | Yes | No | +/− | +/− |
| cp-like4 | XM_317082 | 3 | 2E−15 | 612 | 204 | NO | Yes | No | +/− | +/− |
| cp-like5 | XM_310544 | X | 8E−42 | 2346 | 782 | NO | No | No | +/− | +/− |
| cp-like6 | XM_317386 | 3 | 8E−41 | 1065 | 354 | NO | No | No | +/− | +/− |
| cp-like7 | XM_317389 | 3 | 2E−40 | 747 | 249 | NO | No | No | +/− | +/− |
| cp-like8 | XM_316269 | 2 | 5E−35 | 1011 | 336 | NO | No | No | +/− | +/− |
| cp-like9 | XM_316278 | 2 | 7E−51 | 891 | 297 | NO | No | No | − | − |
| cp-like10 | XM_317391 | 3 | 3E−44 | 1074 | 357 | NO | Yes | No | − | − |
| cp-like11 | XM_317390 | 3 | 5E−38 | 1020 | 339 | NO | Yes | No | − | − |
| cp-like12 | XM_317387 | 3 | 2E−30 | 963 | 320 | NO | No | No | − | − |
| cp-like13 | XM_316276 | 2 | 1E−101 | 1248 | 415 | NO | No | Yes | − | − |

++: Expressed at a significant level;
+: expression detectable on ethidium bromide-stained agarose gel after thirty-five amplification cycles;
+/−: faintly detectable on ethidium bromide-stained agarose gel after thirty-five amplification cycles;
No^{a}: incomplete N-terminal sequence.
CP transcripts are identified by their NCBI accession numbers which matched the Ensembl annotation release 11.2.1 and are accessible at NCBI.

To test whether the predicted genes were transcribed in An. gambiae, pairs of primers specific for each sequence were designed and used in RT-PCR experiments. Expression was analysed in midguts and carcasses of sugar-fed female mosquitoes. Among the genes encoding putative CPB, only cpbAg1 and cpbAg2 were transcribed at significant levels in mosquito midguts (Table 3). cpbAg3 was faintly detectable on ethidium bromide-stained agarose gels, suggesting that it was poorly expressed under the inventors' experimental conditions. The present inventors did not detect any expression of cpbAg4 and cpbAg5, whereas genomic DNA amplification yielded the expected fragments. None of these three latter genes was expressed in the midgut after a blood meal (data not shown). The five predicted CPA encoding genes were expressed in sugar-fed mosquitoes, with cpaAg1 and cpaAg2 specifically expressed in midguts (Table 3). Among the cp-like genes, eight (cp-like1 to cp-like8) were expressed in the mosquito, both in the midgut and carcass, two of them (cp-like1 and cp-like2) being expressed at a significant level. No transcripts could be detected for the remaining ones, even though genomic DNA amplification yielded the expected fragments.

In this Example, the present inventors report the characterization of CPBAg1 and CPBAg2, which are the first insect carboxypeptidases B identified. Using the baculovirus expression system to produce recombinant CPBAg1 the present inventors confirmed the carboxypeptidase B activity of this enzyme. Furthermore, CPBAg1 exhibits a higher specificity towards arginine residues than towards lysine residues, as reported for other carboxypeptidases B (69). The Km of the recombinant enzyme is roughly ten times higher than those reported for vertebrate carboxypeptidases B determined using the same substrates (70-72).

A major function associated with carboxypeptidases in haematophagous insects is blood digestion. Expression of cpbAg1 occurs preferentially in An. gambiae midguts and is down-regulated by a blood meal. However, the expression profile of cpbAg1 differs from those of all carboxypeptidase genes from haematophagous insects examined to date. Indeed, cpbAg1 mRNA abundance increased between day 2 and day 5 post-emergence and was strongly reduced in response to a blood meal as soon as 3 h PBM. In contrast, the An. gambiae carboxypeptidase A (AgCP) gene displays a peak of expression 3 h PBM (54), carboxypeptidase A from S. vittatum (55) and from Ae. aegypti (AeCP) (56) exhibits a peak of expression between 16 and 24 h PBM, whereas the Glossina carboxypeptidase gene is up regulated as early as 1 h PBM (28). In fact, by comparison with the expression patterns of other digestive enzymes, cpbAg1 resembles those of An. gambiae early trypsins (73).

Previous studies provide evidence that early trypsin mRNA accumulates in the midguts of unfed mosquitoes and that the production of the enzyme is regulated at the translational level (74). In addition, early trypsins have been proposed to be part of a signalling pathway that induces expression of late trypsins, responsible for the major phase of digestion (75-77). An analogous role has been suggested for the early chymotrypsin AgChyL (78), which is expressed in unfed females and whose expression drops drastically 8 h PBM. The similarity between the blood meal regulation of cpbAg1, early trypsins and to some extent AgChyL suggests that cpbAg1 might also be involved in the activation of another class of digestive enzymes later during the digestive process. Summarizing the available information regarding the effect of a blood meal on transcriptional regulation of An. gambiae early trypsins (antryp3 to antryp7, 73) and early chymotrypsin (AgChyL) on one hand and An. gambiae carboxypeptidase B (cpbAg1) and carboxypeptidase A (cpaAg1/AgCP) on the other, it is worth noting that (i) genes for early trypsins, which cleave polypeptides at arginine and lysine residues, display the same transcriptional profile as cpbAg1, whose enzyme removes C-terminal arginine or lysine residues; (ii) the gene (AgChyL) for early chymotrypsin which cleaves polypeptides preferentially at phenylalanine residues, is expressed in blood-fed females with the same time laps as cpaAg1/Agcp, whose product removes C-terminal hydrophobic residues including phenylalanine. Thus, in the An. gambiae midgut, the concerted regulation of genes for early endopeptidases (early trypsins and early chymotrypsin) and early exopeptidases (cpbAg1 and cpaAg1/AgCP) likely reflects a finely tuned adaptation of the mosquito to its haematophagous life. Since cpbAg1 is also expressed at a significant level in male midguts and during larval development, with a peak of expression in the second larval instar (C. Lavazec, unpubl. data), it probably serves more general functions in the mosquito life besides being involved in blood digestion.

The An. gambiae genome contains, in addition to cpbAg1, twenty-two sequences predicting zinc-carboxypeptidases with high similarity to cpbAg1. Five predicted proteins have signatures for CPB activity and five others, signatures for CPA activity. Although no predicted protein harbouring features of carboxypeptidase C were detected in the An. gambiae genome (79), this situation is very similar that of Drosophila melanogaster. Indeed, the fly genome contains nineteen genes predicting carboxypeptidases with five predicted carboxypeptidases B and five predicted carboxypeptidases A. Interestingly, all An. gambiae predicted CPBs but one are located on chromosome 2 and all predicted CPAs but one are located on chromosome 3.

In addition, on each of these two chromosomes, the predicted carboxypeptidases are grouped in clusters. On chromosome 3, seven predicted carboxypeptidases are clustered in a region covering only 15 kb. These genes also mapped at the same branch of the phylogenetic tree built on sequence similarity among the An. gambiae zinc carboxypeptidase family (FIG. 15). This strongly suggests that some arose from gene duplication events. Transcripts corresponding to some of them were not detected in the inventors' experimental conditions suggesting that they either represent pseudogenes, or are expressed in other developmental stages. The present inventors cannot exclude that some might be incorrectly annotated. Similarly, all predicted CPs located on chromosome 2 are found in a cluster covering 28 kb. Within this cluster a subcluster covering 9 kb contains cpbAg1 and four other predicted CPs of which only one (cpbAg3) gives rise to a detectable transcript. Their phylogenetic relationship is not as strong as in the former case. They seem, however, to have diverged in only two steps. Only four predicted CPs are located on chromosome X, two of which (cpaAg4 and cp-like2) might have diverged recently following a duplication event, as they cluster in the phylogenetic tree and are separated by only 2 kb on the genome. In addition, both are expressed at a significant level in the mosquito. Except for the CP-encoding genes expressed in the mosquito midgut, all of which are likely to be involved in blood digestion, the significance for the mosquito life of the other CP genes remains to be determined.

In conclusion, this Example represents the first carboxypeptidase B identified in An. gambiae.

### Example 2

### Role of Carboxypeptidase B in Development of P. falciparum in An. gambiae Midgut

Materials and Methods

Field Infection of An. gambiae with P. falciparum

Asymptomatic village people (Senegal) or schoolchildren aged ≦10 years old (Cameroon) were mass-screened to detect parasite carriers as previously described (12). All participants were volunteers and the consent of the children's parents was obtained. The experimental protocols were approved by the Cameroonian and the Senegalese National Ethics Committees. Infections and control experiments were performed with the blood from gametocyte carrier volunteers and with the blood from parasite-free donors. Venous blood (10 ml) was collected in a heparin coated and pre-warmed tube, centrifuged for 5 minutes, 5,000 rpm, at 37° C. and the serum of the patient immediatly replaced with a prewarmed AB serum collected from donors not living in a malaria endemic country. For each experiment, batches of 60 nulliparous female mosquitoes (5 days old) starved from sugar for 24 h were fed for 10 minutes, using the artificial membrane feeding technique (24). Fully engorged females were maintained at the insectarium until dissection. For each experiment a control batch of mosquitoes was used to determine the rate and intensity of infection by oocyst detection on day 7 post feeding. These experiments were performed with a control providing an infection rate close to or above 50%. As described below these basic procedures for infecting An. gambiae with P. falciparum were modified to answer specific issues.

P. berzhei Infections

Swiss mice were peritoneally inoculated with P. berghei gametocyte-producing ANKA 2.34 strain or gametocyte-defective ANKA 2.33 strain. Batches of 60 mosquitoes were fed on infected or non-infected mice for 10 minutes. Fully engorged females were maintained at 21° C., 80% humidity, until dissection. Three independant infection experiments were performed with each strain and three non-infected mice were used as control. The proportion of P. berghei infected mosquitoes on day 11 ranged from 51.7% to 80% for infections with the ANKA gametocyte-producing strain 2.34. As expected, no oocysts were detected on midguts from mosquitoes fed on the ANKA gametocyte-defective strain 2.33.

Gene Expression Analysis

To follow gene expression during blood-meal processing, a batch of nulliparous females (5 days old) was starved from sugar for 24 h and fed on uninfected human blood for 10 minutes. Just before feeding the mosquitoes, red blood cells were centrifuged for 10 minutes at 2,000 rpm, washed three times in RPMI incomplete medium (Gibco), and resuspended in human AB serum. From the fully fed females, midguts were isolated and pools of 40 midguts were made at each 3 h, 6 h, 9 h, 12 h, 15 h, 18 h, 24 h and 48 h post blood-meal (PBM).

To follow gene expression after P. falciparum infection, midguts were isolated from at least 10 females at 14 h, 24 h and 48 h PBM. Three experiments that led to an infection rate of An. gambiae higher than 42% on day 7 were analysed. To discriminate between the biological effect of gametocytes and asexual stages, a series of three experiments was performed with in vitro P. falciparum cultured asexual stages (3D7 strain). Just before feeding the mosquitoes, infected red blood cells were centrifuged for 10 minutes at 2,000 rpm and resuspended in a mixture of washed uninfected red blood cells and human AB serum. A batch of control mosquitoes was fed with the same uninfected red blood cells treated as described above and supplemented with AB serum.

To follow gene expression after P. berghei infection, pools of 20 midguts were dissected at 14 h, 24 h and 48 h PBM for mosquitoes fed on the gametocyte-producing and gametocyte-defective P. berghei strains (See above).

Total RNA was extracted from midguts using the Tri Reagent® kit (M.R.C.Inc) according to the manufacturer's instructions and RNA was treated with the DNA-free® kit (Ambion). Absence of contaminating genomic DNA in each RNA sample was checked by specific amplification of the cpbAg1 gene using the following primers: cpbAg1U: 5′-GGCGGCTGAGGCGTGACT-3′(SEQ ID NO: 5) and cpbAg1L: 5′-GACGGGTCTGATCGACTG-3′(SEQ ID NO: 6). Each RT experiment was performed with 100 ng of RNA incubated with a random hexamer mixture and MMLV reverse transcriptase (400 units per reaction, Invitrogen) in a final volume of 40 μl. To minimize variations during the reverse transcitption step, RT reactions were performed in triplicate and RT products were pooled. Real-time PCR was performed using the dsDNA dye SyberGreen® (MasterMix Perkin Elmer) and the iCycler® apparatus (Bio-Rad). PCR experiments were performed in quintuplicate in 25 μl final volume containing 900 nM of each forward and reverse primer and 5 μl of a ⅕ dilution of the RT product. Relative quantification of cpbAg1 or cpbAg2 mRNA was performed using the standard curve method (User Bulletin 2, ABI) and the ribosomal protein S7 mRNA as endogenous reference. The relative amounts of cpbAg1 mRNA, cpbAg2 mRNA and S7 mRNA was determined from a standard curve constructed using an mRNA sample of known concentration. The primers used for amplification were: S7U: 5′-CACCGCCGTGTACGATGCCA-3′(SEQ ID NO: 7); S7L: 5′-ATGGTGGTCTGCTGGTTCTT-3′(SEQ ID NO: 8); cpbAg2U: 5′-TCCGGCACAATTGGACTACT-3′(SEQ ID NO: 9); cpbAg2L: 5′-TACCGCAGGTACTTGTTGAG-3′(SEQ ID NO: 10); and cpbAg1U and cpbAg1L described above.

Enzymatic Assays on Midgut Extracts

Isolated midguts were homogenized in E buffer (100 mM NaCl/50 mM Hepes/100 μM ZnCl₂ pH 7.2) using a hand-held plastic pestle and centrifuged at 10,000 g, 4° C., for 20 min. Activities were assayed in 20 μl of E buffer containing 1 mM of the dipeptide Hippuryl-Arginine (Sigma) as CPB substrate. The reaction was started by the addition of 20 μl of midgut extract (equivalent to one midgut), incubated at 25° C. for 5 to 40 min, and stopped by the addition of 20 μl of Ninhydrin reagent (Sigma). The rate of the reaction was measured by estimating the released amino acids by the ninhydrin procedure (25). One unit of enzyme activity was defined as μmol of amino acids released/min. For all assays, triplicate reactions were performed.

To assess midgut CPB activity upon ingestion of P. falciparum, pools of 20 midguts were dissected from unfed mosquitoes and from mosquitoes fed on non-infected and P. falciparum gametocyte-containing blood at different times post blood meal (14 h, 24 h, 48 h). Three independent infection experiments were performed and the proportion of P. falciparum infected mosquitoes on day 7 ranged from 30% to 70%.

To assess inhibition of midgut CPB activity upon ingestion of anti-CPBAg1 antibodies, pools of 10 midguts were dissected from mosquitoes fed on uninfected human red blood cells mixed with rabbit serum directed against a recombinant CPBAg1 protein (23) or with a pool of naive rabbit sera, at different times post blood meal (14 h, 24 h, 48 h).

Effect of Carboxypeptidase B Substrates, Free L-Arginine and Free L-Lysine on P. falciparum Development

Mosquito infections were performed with the blood from volunteers harboring a low gametocytaemia, as described above, except that 10 μl of L-arginine (Sigma), L-lysine (Sigma), Hippuryl-Lysine (Sigma) or Hippuryl-Arginine (Sigma) at 10, 1, 0.1 and 0.01 mM in TrisHCl 50 mM pH 7.2 were added to 500 μl of P. falciparum infected blood just before mosquito feeding. As a control 10 μl of TrisHCl 50 mM pH 7.2 was added to the P. falciparum infected blood. Triplicate experiments using the blood of three different gametocyte carriers were performed except for L-lysine where only two experiments could be performed. Rate and intensity of infection were scored on day 7 after blood feeding by detection of oocysts on the mosquito midgut wall.

For the data presented in Table 4 (below), An. gambiae were fed on P. falciparum infected blood supplemented with CPB substrates: Hippuryl-Arginine or Hippuryl-Lysine. Proportion of infected mosquitoes and oocyst counts per positive midgut were determined on day 7 post feeding. For each substrate and concentration tested, data from three independent infections were pooled. Proportions of infected mosquitoes with carboxypeptidase B substrates were significantly different from those generated in control experiments (p<0.05). The infected blood harboured few gametocytes resulting in a low infection rate in the control and thereby allowing the detection of the effect of carboxypeptidase substrates.

For the data presented in Table 5 (below), An. gambiae were fed on P. falciparum infected blood supplemented with L-arginine or L-lysine at three different concentrations. Proportion of infected mosquitoes and oocyst counts per positive midgut were determined on day 7 post feeding. Data were pooled from three independent infections for L-arginine experiments, and from two independent infections for L-lysine experiments. Proportions of infected mosquitoes from L-arginine and L-lysine treatment were significantly different from those obtained in control experiments (p<0.05).

P. falciparum Transmission Blocking Assay

In these series of experiments, the serum of the patient was replaced either with rabbit serum directed against a recombinant CPBAg1 protein (from two different rabbits) or with rabbit immune serum mixed with human AB serum (ratio 1:1). As a control, the serum of the patient was replaced either with a pool of naive rabbit sera, or with a mixture of naive rabbit serum and human AB serum in the same ratio. The volumes of serum required for these experiments precluded the use of the pre-immune rabbit serum. Rate and intensity of infection were scored on day 7 after blood feeding by detection of oocysts on the mosquito midgut wall.

For the data presented in Table 6 (below), An. gambiae were fed on P. falciparum infected red blood cells supplemented with either undiluted or diluted anti-CPBAg1 serum. Proportion of infected mosquitoes and oocyst counts per positive midgut were determined on day 7 post feeding. Infection 1, 2 and 3 were performed with anti-CPBAg1 serum from the same rabbit, whereas infection 4 was performed with anti-CPBAg1 serum from another rabbit. Data from experiments using the anti-CPBAg1 serum were significantly different from those obtained in control experiments (p<0.05).

Statistical Analysis

For gene expression analysis, significant differences in gene expression ratios were evaluated with the Wilcoxon test. For transmission blocking assays and infections with carboxypeptidase substrates or products, significant differences in infection prevalence rates were evaluated by χ² analyses.

Results and Discussion

cpbAg1 and cpbAg2 are Specifically Upregulated upon Ingestion of P. falciparum Gametocytes

The present inventors analyzed the expression pattern of cpbAg1 and cpbAg2 in midguts of mosquitoes fed on Plasmodium infected blood using quantitative RT-PCR in real time. cpbAg1 was initially selected because its expression was modified upon ingestion of P. falciparum (26), while cpbAg2 as selected due to its putative role as a carboxyypeptidase B. Gene expression was monitored at different times post blood-meal corresponding to the transformation of zygotes into ookinetes (14 h), to the interaction of ookinetes with the peritrophic matrix and midgut cells (24 h), and to the migration and early differentiation of ookinetes into oocysts (48 h).

Mosquitoes were first infected by in vitro cultures of P. falciparum asexual stages (3D7 strain, 1,000 asexual stages/μl), which did not harbor gametocytes. No oocyst was detected on day 7 PBM on midguts of mosquitoes fed on theses cultures. Mosquitoes were then infected with blood from volunteers that contained gametocytes but not asexual stages, as assessed by microscopic examination of thick blood smears. The gametocyte loads varied from 50 to 2,600 gametocytes per microliter of blood. The proportion of infected mosquitoes fed on the blood from these gametocyte carriers was greater than 42% on day 7 PBM, with an intensity of infection varying from 1 to 80 oocysts per positive midgut.

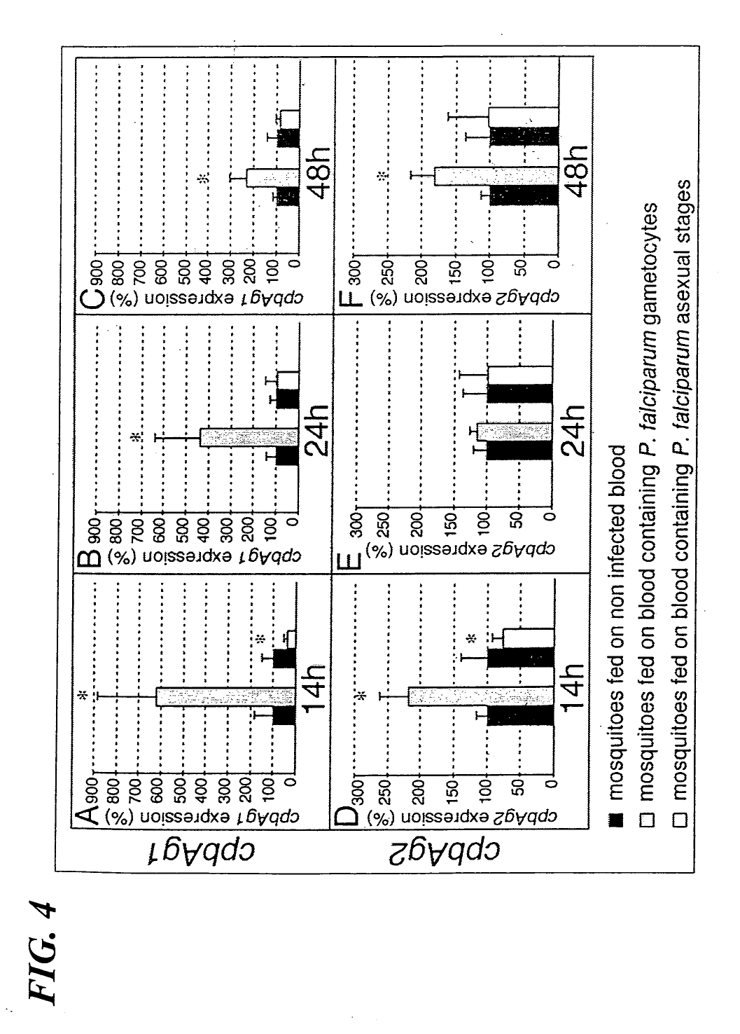

As shown in FIG. 4, expression of cpbAg1 and cpbAg2 differed substantially in mosquitoes fed on P. falciparum gametocytes compared to mosquitoes fed on uninfected blood or on blood containing asexual parasites. cpbAg1 was over-expressed at all time points studied with approximately 6-fold increase above the level detected in mosquitoes fed on non-infected blood at 14 h PBM (FIG. 4A). Its expression then gradually decreased over time, remaining approximately 2-fold above the control level at 48 h PBM (FIGS. 4B-C). In contrast, cpbAg2 was moderately upregulated, again in two waves: at 14 h PBM, where it showed a 2.2 fold over-expression, and at 48 h PBM, with a 1.8 fold over-expression (FIGS. 4D-F). In mosquitoes fed on P. falciparum asexual stages, expression of cpbAg1 and cpbAg2 was slightly reduced 14 h PBM but was similar to that of the control at the other time points. Therefore, expression of both cpbAg1 and cpbAg2 is upregulated in mosquitoes fed on P. falciparum gametocytes, but not in mosquitoes fed on P. falciparum asexual stages.

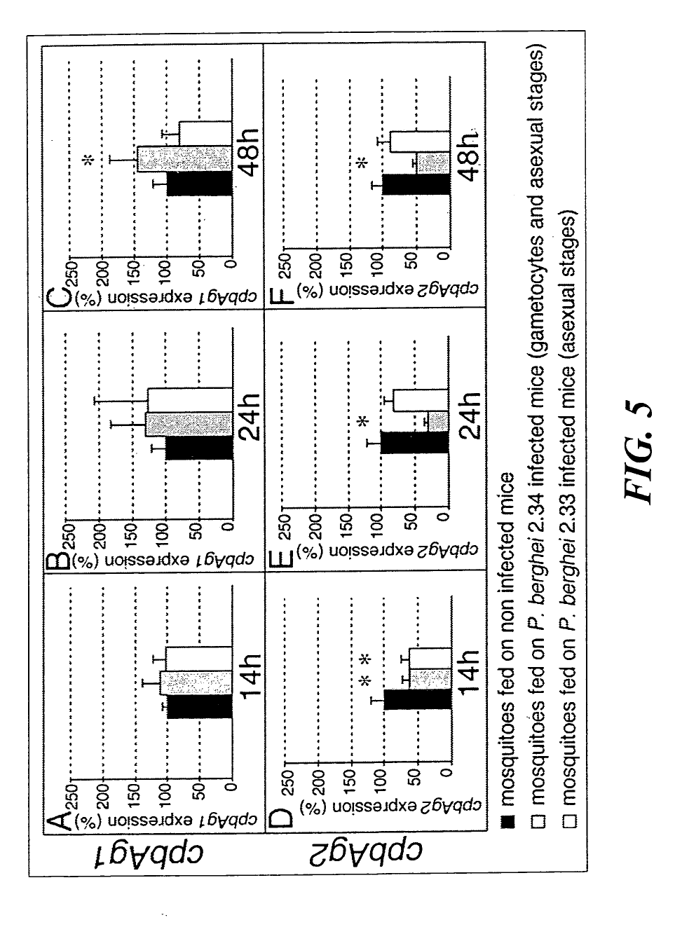

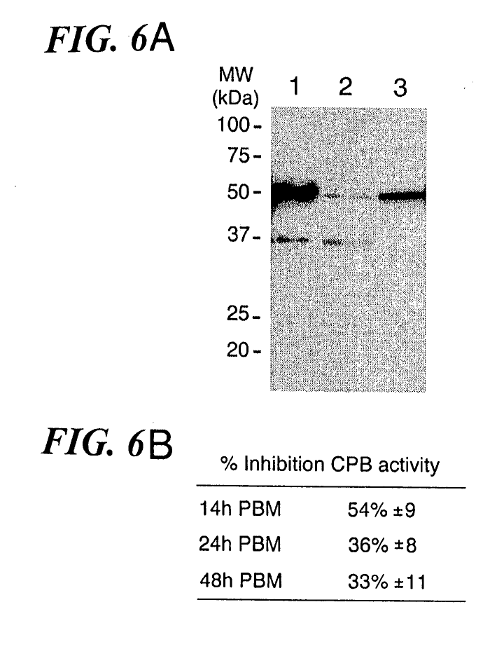

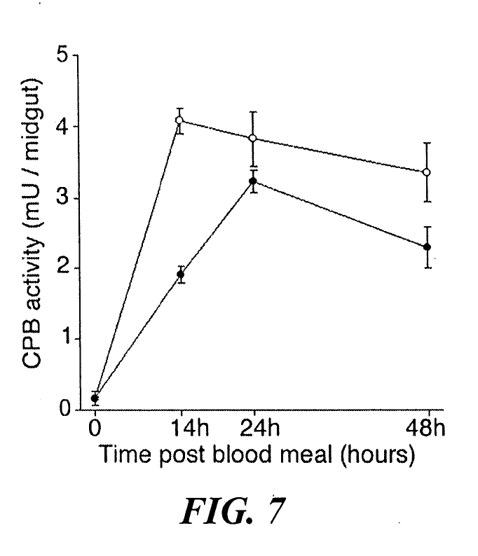

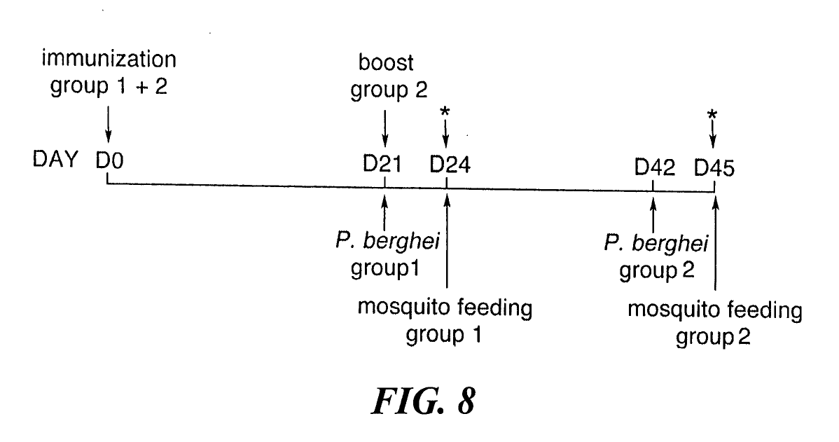

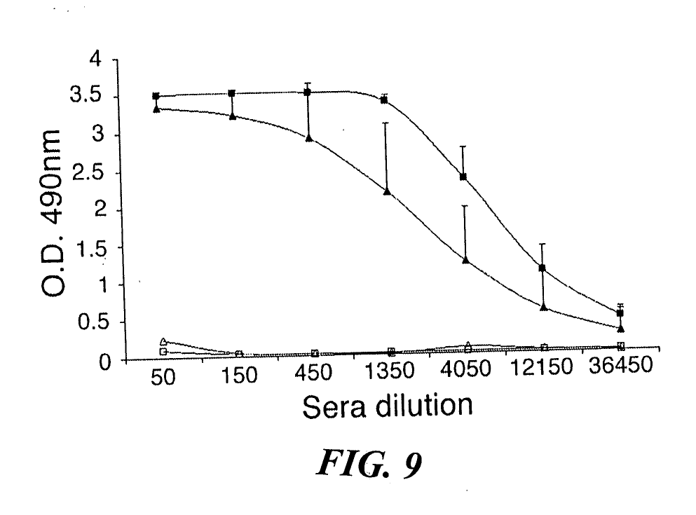

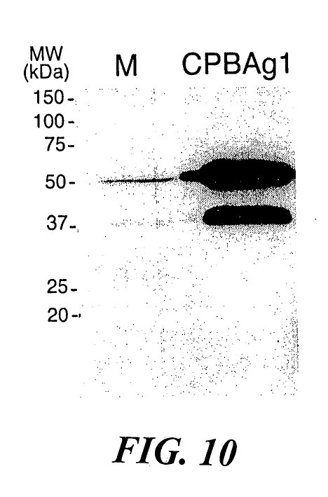

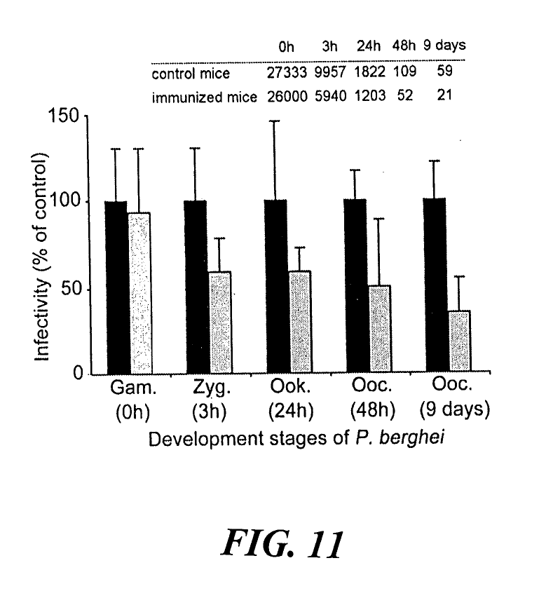

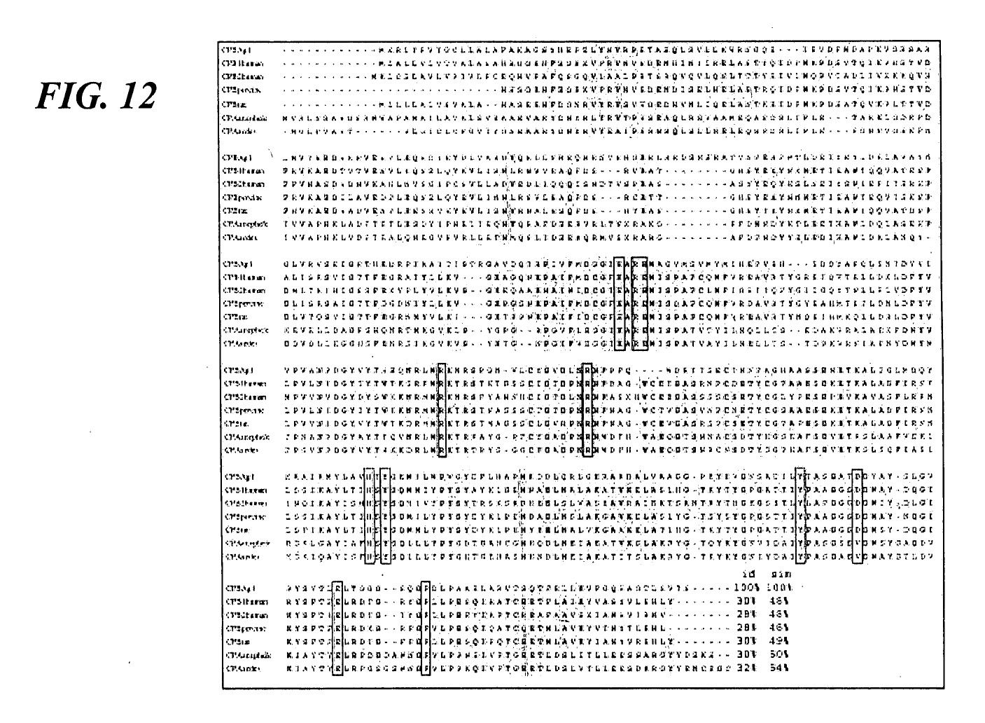

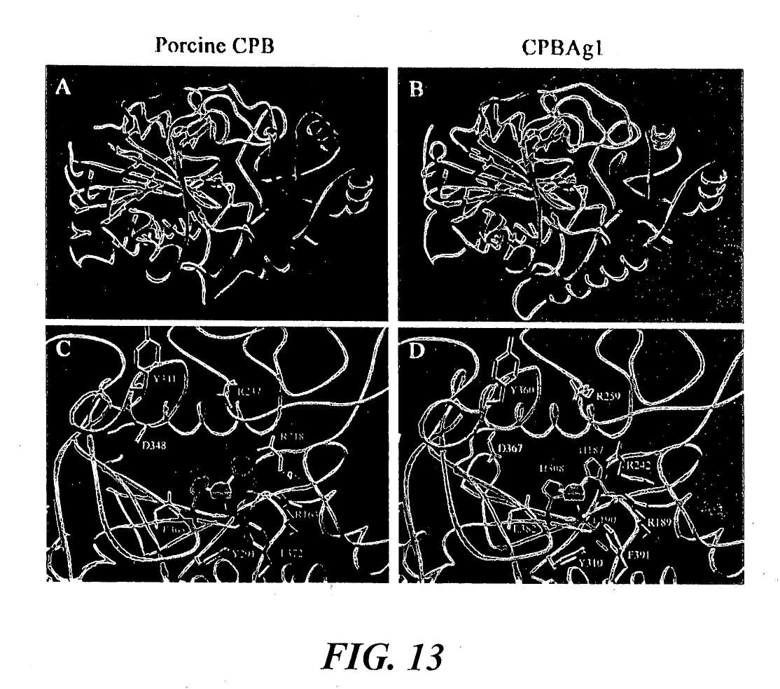

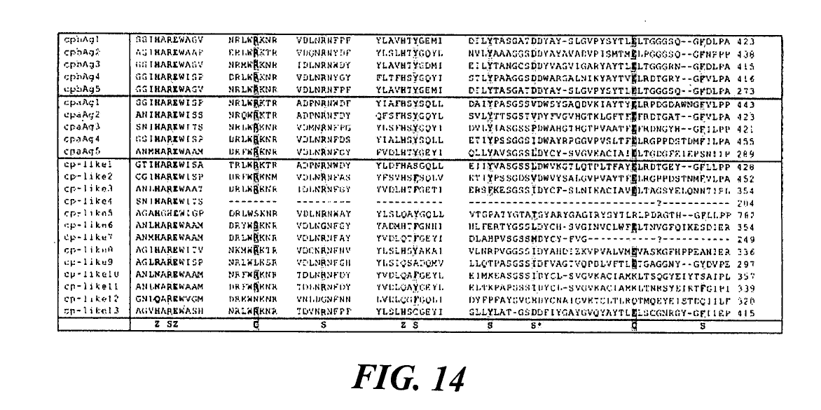

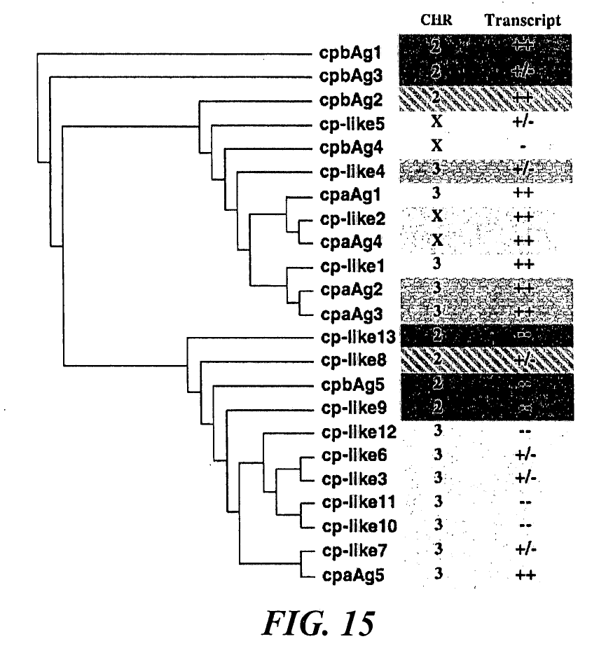

Mosquitoes were next infected with P. berghei, a species that infects rodents and is not naturally transmitted by An. gambiae. Mosquitoes fed on the gametocyte-producing ANKA 2.34 strain or the non-gametocyte-producing ANKA 2.33 strain displayed a similar expression of cpbAg1 as mosquitoes fed on uninfected mice, except at 48 h PBM where cpbAg1 expression was slighlty increased in mosquitoes fed on ANKA 2.34 (FIGS. 5, A-C). In contrast, expression of cpbAg2 appeared to be down-regulated in mosquitoes fed on either ANKA 2.34 from 14 h to 48 h PBM or ANKA 2.33 at 14 h PBM (FIGS. 5, D-F). The strongest effect was observed at 24 h PBM with a 68% reduction of expression when compared to mosquitoes fed on uninfected mice. Taken together these results show that P. falciparum gametocytes specifically upregulate the expression of both carboxypeptidases, particulary cpbAg1, in the midgut of An. gambiae.

P. falciparum Triggers an Increase of Carboxypeiptidase B Activity in An. gambiae Midgut

To determine if the upregulation of cpbAg1 and cpbAg2 in P. falciparum infected mosquitoes correlates with a increased CPB activity in the mosquito midgut, enzymatic activity was assessed in mosquitoes fed on P. falciparum gametocyte-containing blood and on non-infected blood as a control. As depicted in FIG. 7, CPB activity in midguts of mosquitoes fed on P. falciparum gametocytes was greater than that detected in midguts of non-infected mosquitoes. The greatest increase was observed at 14 h PBM with a 113% increase of CPB activity in mosquitoes fed on P. falciparum. The enzymatic activity in infected mosquitoes remained 18% and 39% above the control level at 24 h and 48 h PBM, respectively. In addition, the presence of P. falciparum modified the CPB activity profile as the maximal CPB activity occurred at 14 h PBM whereas it occurred at 24 h PBM in non-infected mosquitoes. These results show that CPB activity in An. gambiae midgut increased upon ingestion of P.falciparum gametocytes, with a peak of activity at 14 h PBM corresponding to the peak of expression observed for the cpbAg1 and cpbAg2 genes.

Carboxypeptidase B Substrates and Products Enhance the Efficiency of P. falciparum Development

The increase in CPB activity upon P. falciparum ingestion suggests that this activity may have an effect on parasite development within the mosquito midgut. Since carboxypeptidases B cleave lysine and arginine residues at the C-terminus of proteins, their activity increases the amounts of free lysine and/or arginine residues in the medium. To test whether CPB activity has an effect on P. falciparum development, the present inventors developed two assays to increase the amount of free arginine or lysine in the midgut lumen, by adding to the gametocyte-containing blood meal the di-peptide Hippuryl-Arginine or Hippuryl-Lysine, as CPB substrates, and free L-arginine or free L-lysine, as CPB products. In order to detect an increase in the rate and intensity of the mosquito infection, experiments were performed with blood from carriers harboring low gametocytemia which gave a low degree of mosquito infection. The results are shown in Table 4.

TABLE 4
Effect of carboxypeptidase B substrates on P. falciparum development in
An. gambiae.

| | | % Infected | Mean number |
|---|---|---|---|
| | | mosquitoes (number of | of oocysts |
| Treatment group | | dissected mosquitoes) | (range) |
| Hippuryl-Arginine | control | 6.7 (74) | 1 (1) |
| | 10 μM | 26.3 (80) | 2.3 (1-10) |
| | 100 μM | 40.9 (88) | 2.1 (1-7) |
| | 1 mM | 28 (50) | 2.1 (1-8) |
| Hippuryl-Lysine | control | 4.3 (70) | 1 (1) |
| | 100 μM | 41.1 (90) | 2.4 (1-6) |
| | 1 mM | 46.7 (92) | 2.5 (1-12) |
| | 10 mM | 23.8 (80) | 2.1 (1-6) |

As shown in Table 4, addition of either Hippuryl-Arginine or Hippuryl-Lysine increased the proportion of infected mosquitoes. Under the inventors' experimental conditions, the increase varied from 4 to 6 fold for Hippuryl-Arginine and from 5.5 to 11 fold for Hippuryl-Lysine. Addition of L-arginine or L-lysine to the infected blood meal also increased the degree of mosquito infection from 2 to 4 fold (Table 5).

TABLE 5
Effect of L-arginine and L-lysine on P. falciparum development in
An. gambiae.

| | | % Infected mosquitoes | mean number |
|---|---|---|---|
| | | (number of | of oocysts |
| Treatment group | | dissected mosquitoes) | (range) |
| L-arginine | control | 13.1 (86) | 1.7 (1-5) |
| | 100 μM | 38.4 (86) | 3.2 (1-13) |
| | 1 mM | 34.4 (64) | 5.3 (1-26) |
| | 10 mM | 55.2 (58) | 5.2 (1-31) |
| L-lysine | control | 15 (40) | 11.5 (1-28) |
| | 10 μM | 62.9 (35) | 8.2 (1-18) |
| | 100 μM | 42.1 (38) | 4.9 (1-15) |
| | 1 mM | 30.5 (36) | 8.3 (1-44) |

In both conditions, no significant effect was detected on the intensity of infection as measured by the number of oocysts detected on the midgut wall on day 7 after ingestion of parasites. These results show that CPB substrates and products enhance the efficiency of P. falciparum development and suggested that CPB activity facilitated P. falciparum development.

Anti-CPBAg1 Serum Inhibits P. falciparum Development

To test further the involvement of CPB activity in facilitating P. falciparum development, the present inventors assessed the effect of inhibiting CPB activity on the development of the parasite in An. gambiae midgut. To this end the present inventors used a rabbit serum raised against a recombinant CPBAg1 protein (23) that recognizes two bands corresponding to the uncleaved (48.2 KDa) and cleaved (37 KDa) CPBAg1 protein (FIG. 6A, lane 1). This serum also recognized a CPBAg2 recombinant protein and two midgut proteins having the expected size and pattern of expression of native CPBAg1 and CPBAg2 (FIG. 6A, lane 2 and 3). A pool of naive rabbit sera used as a control did not recognize any protein in midgut and carcass extracts, further confirming the specificity of the antibodies (not shown).

First, the present inventors tested the ability of anti-CPBAg1 serum to inhibit CPB activity in vivo. As shown in FIG. 6B, the addition of anti-CPBAg1 serum to a non-infected blood meal led to a reduction of CPB activity compared to the control experiments using a pool of naive rabbit sera. The enzymatic activity was inhibited by 54%, 36% and 33% at 14 h, 24 h and 48 h PBM, respectively.

Second, the present inventors assessed the effect of adding anti-CPBAg1 serum to an infected blood meal on P. falciparum development. The results are shown in Table 6.

TABLE 6
Effect of anti-CPBAg1 serum on P. falciparum development in An. gambiae.

| | % infected mosquitoes | | | | mean number of oocysts | | | | |
|---|---|---|---|---|---|---|---|---|---|
| Experimental | (Number of dissected mosquitoes) | | | | (range) | | | | % of |
| conditions | infection 1 | infection 2 | infection 3 | infection 4 | infection 1 | infection 2 | infection 3 | infection 4 | reduction^{a} |
| non diluted serum | | | | | | | | | |
| control | 72.2 (18) | 82.4 (34) | 46.6 (30) | nd | 2.5 (1-6) | 57.9 (1-150) | 2.9 (1-11) | nd | |
| Exp | 0 (30) | 6.7 (30) | 0 (30) | nd | 0 | 1.5 (1-2) | 0 | nd | 96.7 |
| diluted serum | | | | | | | | | |
| control | 46.6 (30) | 86.6 (30) | 77.7 (27) | 45 (20) | 4 (1-8) | <80 (1-200) | 12.5 (1-61) | 4.4 (1-10) | |
| Exp | 13.6 (22) | 0 (30) | 0 (30) | 5 (20) | 2 (1-3) | 0 | 0 | 2 (2) | 92.7 |

^{a}% reduction was calculated as R % = 100 × [1 − (% anti-CPBAg1 infected mosquitoes/% infected control mosquitoes)], using the mean value from each series of four infections.

As shown in Table 6, the addition of anti-CPBAg1 serum to a P. falciparum gametocyte-containing blood meal blocked the development of the parasite leading to a reduction of at least 92% in the number of infected mosquitoes on day 7 post infection compared to the control experiments using a naive rabbit serum. These results were consistently obtained in four independent infection experiments using either undiluted or diluted anti-CPBAg1 rabbit serum from two different rabbits. In addition, the small number of infected mosquitoes fed on anti-CPBAg1 antibodies harbored very few oocysts compared to the control groups fed on the same infected blood supplemented with naive rabbit serum.

Therefore, the serum directed against CPBAg1 inhibits CPB activity and exhibits a blocking-effect against P. falciparum development in An. gambiae midgut suggesting that CPB activity is required for parasite development.

In this example, the present inventors have identified a novel carboxypeptidase encoding gene, cpbAg1, from the malaria vector Anopheles gambiae, and provide evidence for a role of its product in P. falciparum development in An. gambiae midgut. The present inventors showed that both cpbAg1 and cpbAg2, which also encodes a carboxypeptidase B, are mainly expressed in midguts of An. gambiae and that their expression is upregulated upon ingestion of P. falciparum gametocytes. Such a regulation was not observed when mosquitoes ingested either P. falciparum asexual stages or P. berghei parasites. As the serum of the P. falciparum carriers was replaced with serum from blood of individuals not living in a malaria endemic country, this effect appears to be associated with the presence of P. falciparum gametocytes in the ingested blood.

The data reported here show that CPB activity modulates P. falciparum development in An. gambiae midgut. Indeed, addition of CPB substrates or products of enzymatic activity to an infected blood meal enhanced the efficiency of P. falciparum development, suggesting that CPB activity favors the successful development of P. falciparum within An. gambiae midgut. Conversely, adding antibodies directed against CPBAg1 to a P. falciparum-containing blood meal inhibited CPB activity and blocked the development of the parasite in the mosquito midgut, providing evidence that CPB activity is required for the parasite successful development.

Moreover, providing carboxypeptidase B substrates or arginine, a product of carboxypeptidase activity, to an infected blood-meal strongly enhanced the efficiency of P. falciparum development, providing additional evidence that carboxypeptidase B activity favors the successful development of P. falciparum within An. gambiae midgut.

A major function associated with proteases and therefore carboxypeptidases in hematophagous insects is blood digestion. Expression of both cpbAg1 and cpbAg2 occur preferentially in An. gambiae midguts and are regulated by a blood-meal. However, expression profiles of cpbAg1 and cpbAg2 differed from those of all carboxypeptidases from haematophagous insects examined to date. Indeed, expression of known carboxypeptidases from various haematophagous insects is induced upon blood feeding (28, 54-56), whereas cpbAg1 mRNA abundance was strongly reduced in response to a blood-meal as soon as 3 h PBM.

In contrast, expression of cpbAg2 is upregulated in two waves after blood feeding with two peaks of expression at 6 h and 18 h PBM respectively, suggesting that CPBAg2 is involved later in the digestive process. Here, the present inventors show that expression of both cpbAg1 and cpbAg2 are upregulated upon ingestion of P. falciparum gametocytes and this upregulation is greatest at 14 h PBM for both genes. This effect appears to be due solely to the presence of P. falciparum gametocytes and not to seric factors in the ingested blood as the serum of the P. falciparum carriers was replaced with serum from blood of individuals not living in a malaria endemic country. The dosage of enzymatic activity within the mosquito midgut show that ingestion of P. falciparum gametocytes not only triggers an early production of CPB enzyme at a time correspondings to the highest over-expression of cpbAg1 and cpbAg2 but also increases the overall CPB activity for at least 48 h PBM.

Expression of cpbAg1 and cpbAg2 parallels that of early and late trypsin, the most documented mosquito enzymes involved in blood digestion. The fine tuning between both early and late trypsin and carboxypeptidase expression likely provides an efficient means for blood digestion. As both cpbAg1 and cpbAg2 are also expressed at a significant level in male midguts, they probably serve other more general functions in the adult life beside being involved in blood digestion.

Midgut expression of both cpbAg1 and cpbAg2 are induced during the development of P. falciparum but not during the development of P. berghei. On the contrary, the P. berghei gametocyte-producing strain repressed expression of cpbAg2. As the present inventors used infection conditions that were as similar as possible in the two parasite systems, these differences are likely to be due to differences in the interaction of An. gambiae with the two parasite species. These data provide another example that mosquito gene expression can significantly differ between the naturel system An. gambiae/P. falciparum and the model system An. gambiae/P. berghei, as the present inventors previously reported (12).

The modification of midgut enzymatic activity of carboxypeptidase A, another digestive carboxypeptidase, was not observed in Anopheles stephensi infected with the rodent parasite Plasmodium yoelii (27). In contrast, the up-regulation of a midgut specific carboxypeptidase gene has been reported in Tsetse flies infected with Trypanosoma brucei (28). To the knowledge of the present inventors, there has been no other report on the modification of carboxypeptidase expression during the development of midgut parasites in Diptera.

What could be the role of An. gambiae carboxypeptidases during P. falciparum development? Without being limiting in mechanism or function, two main hypotheses are attractive: CPBAg1 and CPBAg2 act directly or indirectly on the parasite. Since the addition of carboxypeptidase products enhanced P. falciparum development, it is likely that CPBAg1 and CPBAg2 do not act directly on the parasite.

Carboxypeptidases B remove lysine or arginine residues from the carboxy-terminus of proteins and these two amino acids are presumably central for the development of the parasite. Indeed, L-arginine can be hydrolyzed by arginase to urea and L-ornithine, which is a precursor for the synthesis of polyamines via the ornithine decarboxylase (ODC) pathway. The inhibition of the ODC pathway inhibits the erythrocytic schizogony of P. falciparum and the sporogonic development of P. berghei in An. stephensi (29, 30). Therefore, L-arginine may favor Plasmodium growth, presumably as a precursor for the synthesis of polyamines which play an important role in regulating the cell cycle of the malaria parasite (31).

As the genome of P. falciparum seems to be devoid of CPB, the parasite can obtain its requirements for lysine and arginine either through the action of endogenous aminopeptidases or by absorbing amino acids from the mosquito. Only one aminopeptidase with the appropriate specificity has been described in P. falciparum (32) and it expression has not been reported in the sporogonic stages of the parasite. Therefore, an attractive role for the mosquito carboxypeptidases in the development of P. falciparum relies on their ability to complement the need of the parasite in L-lysine and L-arginine.

L-arginine and L-lysine are essential amino acids not only for the parasite but also for the mosquito (33). For example, arginine kinase catalyzes the production of phospho-arginine, which is the principal reserve of high energy phosphate compounds in insect muscle (34).

These amino acids are also important for triggerring vitellogenesis (Raikhel, personal communication). P. falciparum and An. gambiae probably compete for these amino acids produced in the mosquito migut lumen. The consumption of free arginine and free lysine by P. falciparum might therefore induce a depletion in the arginine and lysine pools needed for the mosquito that could be counterbalanced by upregulating CPB encoding genes, as described in this report.

Addition of anti-CPBAg1 serum to an infective blood meal led to a drastic reduction of P. falciparum development in the mosquito midgut. This is likely due to the partial inhibition of CPB activity that in turn reduced the available resources in L-arginine and L-lysine below the threshold level needed for parasite development. As the blocking effect of anti-CPBAg1 was observed on field populations of P. falciparum, these data lead the inventors to propose that CPBAg1, and possibly CPBAg2, are good candidate molecules for a P. falciparum transmission blocking vaccine.

In conclusion, the inventors' data show that An. gambiae enhances the expression of two An. gambiae midgut carboxypeptidase B genes and that carboxypeptidase B activity is critical for the successful development of P. falciparum in the midgut of this mosquito. This data constitutes a novel detailed report on the characterisation of An. gambiae molecules involved in the successful development of the human malaria parasite. Importantly, the ability of antibodies against carboxypeptidase to reduce the infectivity of P. falciparum in An. gambiae makes midgut carboxypeptidases B, the first An. gambiae candidate proteins for a P. falciparum transmission blocking vaccine based on mosquito molecules.

### Example 3

### CPBAg1 Immunization

Materials and Methods

Mice and Immunizations

Immunizations were performed with a recombinant CPBAg1 protein expressed in insect cells using the baculovirus expression system. The immunization protocol for the transmission blocking assays is shown in FIG. 8. Eleven 3- to 4-week-old female Swiss outbred mice (CERJ, Le Genest St-Isle, France) were injected subcutaneously with 20 μg of recombinant CPBAg1 in sterile PBS, emulsified with complete Freund's adjuvant (Sigma). Eleven control mice were injected simultaneously with sterile PBS/adjuvant following the same protocol. On day 21 post-immunization, eight immunized mice and eight control mice (group 1) were challenged with P. berghei for transmission blocking assays (see below). The three other immunized mice were boosted with 10 μg of recombinant CPBAg1 formulated in incomplete Freund's adjuvant (Sigma) and the three other control mice were injected simultaneously with sterile PBS/incomplete adjuvant. These six mice, which constituted group 2, were challenged with P. berghei 21 days after the booster immunization. Sera from immunized and control mice of both groups were prepared from 100 μl blood samples collected the same day as mosquito feeding (24 days after the first and booster immunizations).

Enzyme-Linked Immunosorbent Assays (ELISAs)

Microtiter plates (Immuno-plate Maxisorp, Nunc) were coated with 50 ng/well of CPBAg1 in PBS, overnight at 4° C., and saturated with 100 μl/well 0.5% gelatine (Sigma) in PBS for 1 h. Plates were then incubated for 90 min. with serial dilutions (1:50 to 1:36,450) of sera from immunized mice diluted in 0.5% gelatine/0.1% Tween 20 (Merck)/PBS. Plates were washed extensively and incubated with a 1:1,000 dilution of goat anti-mouse immunoglobin G conjugated to horseradish peroxidase (Bio-Rad). After washes, the plates were developed with 100 μl/well of 0.4 mg/ml o-Phenylenediamine (OPD) (Sigma) in 0.05 M phosphate-citrate buffer pH 5 and 0.1% H₂0₂. After 10 min. incubation, the reaction was stopped with 50 μl 3N HCl and absorbance was measured at 490 nm using a microplate reader (Molecular Devices). Serum dilutions at an absorbance value of 0.5 were designated as the endpoint of ELISA titers.

Analysis of Antibody Recognition of Mosquito Midgut Proteins by Western-Blot

Proteins were prepared from mosquito midguts by tissue homogenization in 25 mM Tris-HCl pH 8 using a plastic pestle and further centrifugation at 20,000 g, 4° C., for 15 min. Soluble protein fractions and the recombinant CPBAg1 protein were run on a 12% SDS-poly-acrylamide gel under reducing conditions and blotted onto a PVDF membrane (Hybond-P, Amersham). After saturation in PBS containing 5% skim milk and 0.1% Tween 20, the membrane was incubated with each mouse sera (dilution 1:500). Antibody binding was detected by incubating the membrane with mouse anti-rabbit peroxidase-conjugated antibody (SantaCruz, dilution 1:105) and subsequent treatment with a peroxidase chemiluminescent substrate (SuperSignal Ultra®, Pierce).

Transmission-Blocking Assays

On day 21 after the first and booster immunizations, mice were peritoneally inoculated with the P. berghei ANKA strain 2.34. On day 3 after P. berghei inoculation, parasitemia and gametocytemia were determined using Giemsa stained thin blood smears, and the mice were used for mosquito feeding. In group 1, parasitemia and gametocytemia ranged from 3.2% to 6.2% and from 6,500 to 11,500 gametocytes/μl for control mice, whereas they ranged from 3% to 7.4% and from 7,500 to 13,500 gametocytes/μl for immunized mice. In group 2, they ranged from 2% to 2.9% and from 2,000 to 4,000 gametocytes/μl for control mice, whereas they ranged from 1.5% to 3.6% and from 1,000 to 2,000 gametocytes/μl for immunized mice. Each mouse was placed onto a netted cage containing 50 starved 5-day-old An. gambiae females and feeding was carried out for 15 minutes at 21° C. After mosquito feeding, mice were bled to collect sera for ELISAs experiments and fully engorged mosquito females were maintained at 21° C., 80% humidity, until dissections. Rate of infection (number of infected mosquitoes/number of dissected mosquitoes) and intensity of infection (mean number of oocysts per positive midgut) were scored on day 9-11 after blood feeding by detection of oocysts on the mosquito midgut wall.

For the data reported in Table 7 below, An. gambiae were fed on control or immunized mice from group 1 (after primary immunization) and group 2 (after booster immunization). Proportion of infected mosquitoes (rate of infection) and mean number of oocysts per positive midgut (intensity of infection) were determined on day 9-11 post feeding. R: % reduction was calculated as R %=100×[1−(% infected mosquitoes fed on immunized mice/% infected mosquitoes fed on control mice)], using the mean value from each experimental group.

Effect of CPBAG1 Immunized Mice Sera on Parasite Development

To assess the effect of anti-CPBAg1 sera on parasite development, a new group of six mice was immunized as described above and six control mice were injected with PBS/adjuvant. These mice were peritoneally inoculated 21 days later with the P. berghei PbGFPCON ANKA strain, which constitutively expresses GFP throughout the entire parasite life cycle (49). On day 3 after P. berghei inoculation, gametocytemia were determined by Giemsa staining of thin blood smears and by GFP fluorescence detection. Batches of 100 mosquitoes were fed on individual mouse as described above and pools of 10 to 30 midguts from each batch were dissected 3 h, 24 h, 48 h and 9 days after blood feeding to visualize zygotes, ookinetes, young oocysts and mature oocysts, respectively. Intensity of infection was scored by counting the number of zygotes and ookinetes within the mosquito midgut content, and the number of oocysts on the midgut wall. GFP fluorescence was visualized using GFP filter settings with a Zeiss Axiovert 25 fluorescence microscope.

Statistical Analysis

Analysis of the statistical significance of the difference between infected mosquitoes resulting from feedings on immunized and control mice was performed by the chi-square test. Statistically significant differences in the geometric mean oocyst intensities in mosquitoes fed on immunized and control mice were determined by the F test and ANOVAs.

Results and Discussion

CPBAg1 Triggers the Production of High Titer Antibodies in Mice

As antibodies directed against the An. gambiae carboxypeptidase B CPBAg1, when fed to mosquitoes in membrane feeding assays, exhibit a blocking effect against P. falciparum development in An. gambiae, the present inventors undertook a study to investigate whether CPBAg1 could be a component of a Plasmodium transmission-blocking vaccine. To evaluate the immunogenicity of the An. gambiae CPBAg1 as a vaccine candidate, mice were immunized with the recombinant CPBAg1 protein produced in the baculovirus/insect cells expression system, under two regimens: single injection (group 1, FIG. 8) and two injections at 3 weeks interval (group 2, FIG. 8). As shown in FIG. 9, all mice having received a single injection developed high titers of specific anti-CPBAg1 antibodies 24 days after immunization (endpoint titer ˜12,150), whereas the control mice injected with adjuvant and PBS alone showed marginal CPBAg1-binding antibody levels, indicating that CPBAg1 is immunogenic even after a single injection. The endpoint titer of the sera from mice having received a second injection on day 21 after the first one (group 2) showed higher titers of antibody (endpoint titer ˜36,450).

The specificity of all the immunized mice sera was assessed by western-blot analysis on mosquito midgut extracts (FIG. 10). All the sera specifically recognized two bands in mosquito midgut extracts that likely correspond to the zymogen (37 kDa) and the mature protein (50 kDa) of CPBAg1 as previously described. As expected, the pooled sera from control mice did not detect any specific antigen (not shown).

CPBAG1 Immunized Mice Exhibit Plasmodium Transmission-Blocking Immunity

In order to determine whether the antibodies generated by immunization were functionally effective in blocking the transmission of P. berghei, mosquitoes were fed on immunized and control mice. On day 3 after parasite injection, parasitemia and gametocytemia were in the same range in control mice and immunized mice within each experimental group (see Table 7).

TABLE 7
Transmission-blocking assays

| control mice | | | immunized mice | | | |
|---|---|---|---|---|---|---|
| | Rate of infection % | | | Rate of infection % | | % of |
| Mouse | (number of dissected | Mean no. of | Mouse | (number of dissected | Mean no. of | reduction |
| n^{o} | mosquitoes) | oocysts (range) | n^{o} | mosquitoes) | oocysts (range) | R |
| Group 1 | | | | | | |
| C 1 | 93.3 (30) | 8.4 (1-55) | Im 1 | 10.5 (38)* | 3.3 (1-15) | |
| C 2 | 78.8 (33) | 4.7 (1-56) | Im 2 | 9.4 (32)* | 2.5 (1-5)* | |
| C 3 | 80.6 (31) | 5.8 (1-80) | Im 3 | 0 (20)* | 0* | |
| C 4 | 71.8 (32) | 2.6 (1-27) | Im 4 | 43.3 (30)* | 7.16 (1-56) | |
| C 5 | 80.6 (31) | 7 (1-45) | Im 5 | 31.2 (32)* | 3.7 (1-70) | |
| C 6 | 75.8 (29) | 12.7 (1-91) | Im 6 | 41.2 (17)* | 5.4 (1-27) | |
| C 7 | 62.5 (32) | 3.7 (1-116) | Im 7 | 38.8 (18)* | 5.8 (1-32) | |
| C 8 | 67.9 (28) | 7.4 (1-56) | Im 8 | 50 (30)* | 6.7 (1-117) | |
| Mean | 76.4 (246) | 5.9 | | 28.1 (217)* | 5.3 | 63.2 |
| Group 1 | | | | | | |
| Group 2 | | | | | | |
| C 9 | 96.6 (30) | 24.1 (1-160) | Im 9 | 77.4 (31)* | 23.7 (1-120) | |
| C 10 | 97 (33) | 26.6 (1-180) | Im 10 | 26.6 (30)* | 6.2 (3-10)* | |
| C 11 | 93.3 (30) | 21.8 (1-130) | Im 11 | 36.6 (30)* | 24.3 (6-100) | |
| Mean | 95.7 (93) | 24.2 | | 47.2 (91)* | 18.6* | 50.6 |
| Group 2 | | | | | | |

*statistically significant (p < 0.05) compared to the mean value from control group, determined by the chi-square test for the rate of infection and by the F test and ANOVAs for the intensity of infection.

As shown in Table 7, the rate of infection was significantly reduced in mosquitoes fed on all immunized mice from both groups compared to the mosquitoes fed on mice injected with adjuvant and PBS alone. In addition, the transmission-blocking immunity (TBI) was 10 effective as early as three weeks after the first immunization. In group 1, the immunization with CPBAg1 led to a mean reduction of 63% in the number of infected mosquitoes. The rate of infection was drastically reduced in mosquitoes fed on mice Im 1 and Im 2, and the transmission of the parasite was totally blocked in mosquitoes fed on mouse Im 3. In group 2, the mean reduction in the number of infected mosquitoes fed on immunized mice was lower 15 than in group 1 reaching only 51%. As mice from group 2 presented a higher antibody titer than mice from group 1, which suggests that efficiency of TBI does not correlate with anti-CPBAg1 antibody concentrations. Therefore, effective TBI does not require a booster immunization and an immunogenic response with antibody titers higher than 12,000. Looking at the intensity of infection, the mean numbers of oocysts per positive midgut were significantly reduced in only three experiments (mouse Im 2, mouse Im3 and mouse Im 10). However, it is difficult to observe a significant reduction because of the low mean number of oocyst per midgut in the control groups (from 2.6 to 26.6).

The present inventors have previously described that the addition of a rabbit anti-CPBAg1 serum to a P. falciparum gametocyte-containing blood meal, when fed to mosquitoes in membrane feeding experiments, led to a reduction of at 95% in the number of infected mosquitoes. In the present study, the mouse anti-CPBAg1 antibodies were less effective in inhibiting the sporogonic development of P. berghei. Differences in transmission-blocking efficiencies have been previously described between in vitro and in vivo mosquito feeding experiments (44). However, other explanations for this difference are possible. Firstly, the CPBAg1 recombinant protein used in the inventors' previous study was fused to GST and produced in E. coli whereas a His-tagged recombinant CPBAg1 produced in insect cells was used to immunize mice in the present study. The present inventors cannot exclude that the two recombinant proteins elicit antibodies directed against different epitopes and having different transmission-blocking efficiencies. Secondly, several findings indicate that human and rodent parasites display different interactions with the mosquito midgut. Indeed, the developmental kinetics in the mosquito (50-51), the mode of ookinete migration across the midgut epithelium (52-53), and the expression of immune responsive genes (12) appears to be different in the two Plasmodium species. More importantly, the present inventors previously described that the expression of cpbAg1 gene was upregulated in the midgut upon ingestion of P. falciparum gametocytes, but not by P. berghei. Therefore it is possible that CPBAg1, which is probably involved in blood digestion in the mosquito, does not display exactly the same interactions at the same time with the two Plasmodium species.

Plasmodium Development is Reduced at Different Parasite Life Stages

To determine when and where the parasite development is inhibited in the presence of anti-CPBAg1 sera, six mice were immunized with CPBAg1 and six mice were injected with adjuvant as a control. All these mice were challenged with a P. berghei GFP-expressing strain. Midgut content and midgut wall from mosquitoes fed on control and immunized mice were examined 3 h, 24 h, 48 h and 9 days post blood meal (PBM) to score intensity of infection at different life stages of the parasite (FIG. 11). Just before feeding, no significant differences were noted in gametocytemia in either immunized or control mice. In mosquitoes fed on immunized mice, the present inventors observed that parasite development was not blocked at a particular life stage but that parasite densities decreased at different life stages until day 9 PBM compared to the control experiments. As soon as 3 h PBM, the mean of infection intensity in mosquitoes fed on immunized mice was 40% lower than that in mosquitoes fed on control mice, and this reduction reached 65% at day 9 PBM.

These findings contrast with previous studies demonstrating that antibodies against mosquito midgut extracts specifically targeted the ookinete-oocyst transition (2, 44). The inventors' results suggest that antibodies against CPBAg1 inhibit a physiological process of the mosquito, which is essential for parasite development rather than interfering with a particular parasite-midgut interaction such as ookinete penetration across the midgut epithelium. This observation is consistent with the inventors' previous hypothesis that CPBAg1 releases arginine residues from blood meal proteins that are essential to the parasite as precursors for the synthesis of polyamines, which have multiple roles in regulating parasite cell growth and differentiation (31).

In this Example, the present inventors demonstrate mouse immunization with a recombinant CPBAg1 protein can elicit potent Plasmodium transmission-blocking antibodies as early as three weeks after a single injection. This provides strong support to make CPBAg1 a component of a Plasmodium transmission-blocking vaccine. These data constitute the first report on a transmission blocking vaccine based on a defined antigenic molecule from An. gambiae.

Numerous modifications and variations on the present invention are possible in light of the above teachings. It is, therefore, to be understood that within the scope of the accompanying claims, the invention may be practiced otherwise than as specifically described herein.

### REFERENCES

1. Kaslow, D. C. (1997) Int J Parasitol 27, 183-9.
2. Lal, A. A., Patterson, P. S., Sacci, J. B., Vaughan, J. A., Paul, C., Collins, W. E., Wirtz, R. A. & Azad, A. F. (2001) Proceedings of the National Academy of Sciences of the United States of America 98, 5228-33.
3. Ramasamy, M. S., Kulasekera, R., Wanniarachchi, I. C., Srikrishnaraj, K. A. & Ramasamy, R. (1997) Medical & Veterinary Entomology 11, 290-6.
4. Ito, J., Ghosh, A., Moreira, L. A., Wimmer, E. A. & Jacobs-Lorena, M. (2002) Nature 417, 452-5.
5. Dinglasan, R. R., Fields, I., Shahabuddin, M., Azad, A. F. & Sacci, J. B., Jr. (2003) Infect Immun 71, 6995-7001.
6. Gouagna, L. C., Mulder, B., Noubissi, E., Tchuinkam, T., Verhave, J. P. & Boudin, C. (1998) Tropical Medicine & International Health 3, 21-8.
7. Vaughan, J. A., Noden, B. H. & Beier, J. C. (1994) American Journal of Tropical Medicine and Hygiene 51, 233-243.
8. Luckhart, S., Vodovotz, Y., Cui, L. W. & Rosenberg, R. (1998) Proceedings of the National Academy of Sciences, U.S.A. 95, 5700-5705.
9. Richman, A. M., Dimopoulos, G., Seeley, D. & Kafatos, F. C. (1997) EMBO Journal 16, 6114-6119.
10. Dimopoulos, G., Seeley, D., Wolf, A. & Kafatos, F. C. (1998) EMBO Journal 17, 6115-6123.
11. Oduol, F., Xu, J. N., Niare, O., Natarajan, R. & Vernick, K. D. (2000) Proceedings of the National Academy of Sciences, U.S.A. 97, 11397-11402.
12. Tahar, R., Boudin, C., Thiery, I. & Bourgouin, C. (2002) Embo J 21, 6673-80.
13. Osta, M. A., Christophides, G. K. & Kafatos, F. C. (2004) Science 303, 2030-2.
14. Feldmann, A. M., Billingsley, P. F. & Savelkoul, A. (1990) Parasitology 101, 193-200.
15. Garcia, G. E., Wirtz, R. A. & Rosenberg, R. (1997) Molecular and Biochemical Parasitology 88, 127-135.
16. Billker, O., Lindo, V., Panico, M., Etienne, A. E., Paxton, T., Dell, A., Rogers, M., Sinden, R. E. & Morris, H. R. (1998) Nature 392, 289-292.
17. Gass, R. F. (1977) Acta Tropica 34, 127-140.
18. Gass, R. F. & Yeates, R. A. (1979) Acta Tropica 36, 243-252.
19. Sieber, K.-P., Huber, M., Kaslow, D., Banks, S. M., Torii, M., Aikawa, M. & Miller, L. H. (1991) Experimental Parasitology 72, 145-156.
20. Shahabuddin, M. & Kaslow, D. C. (1994) Experimental Parasitology 79, 85-88.
21. Grotendorst, C. A. & Carter, R. (1987) J Parasitol 73, 980-4.
22. Sinden, R. E., Alavi, Y. 1. H., Butcher, G. A., Dessens, J. T., Raine, J. D. & Trueman, H. E. (2004) in Malaria parasites: Genomes and Molecular Biology, ed. Waters, A. P. a. J., C. J. (Caister Academic Press, Wymondham, UK), pp. 475-500.
23. Lavazec, C., Bonnet, S., Thiery, I., Boisson, B. & Bourgouin, C. (2005) Insect Mol Biol in press.
24. Tchuinkam, T., Mulder, B., Dechering, K., Stoffels, H., Verhave, J. P., Cot, M., Carnevale, P., Meuwissen, J. H. E. T. & Robert, V. (1993) Tropical Medicine and Parasitology 44, 271-276.
25. Moore, S. & Stein, W. H. (1948) J Biol Chem 176.
26. Bonnet, S., Prevot, G., Jacques, J. C., Boudin, C. & Bourgouin, C. (2001) Cell Microbiol 3, 449-58.
27. Jahan, N., Docherty, P. T., Billingsley, P. F. & Hurd, H. (1999) Parasitology 119 (Pt 6), 535-41.
28. Yan, J., Cheng, Q., Li, C.-B. & Aksoy, S. (2002) Insect Mol Biol 11, 57-65.
29. Bitonti, A. J., McCann, P. P. & Sjoerdsma, A. (1987) Exp Parasitol 64, 237-43.
30. Gillet, J. M., Charlier, J., Bone, G., Mulamba, P. L., Bown, D. P., Wilkinson, H. S. & Gatehouse, J. A. (1983) Exp Parasitol 56, 190-3.
31. Bachrach, U. & Abu-Elheiga, L. (1990) Eur J Biochem 191, 633-7.
32. Allary, M., Schrevel, J. & Florent, I. (2002) Parasitology 125, 1-10.
33. Clements, A. N. (1992) The biology of mosquitoes (Chapman and Hall, London).
34. Schneider, A., Wiesner, R. J. & Grieshaber, M. K. (1989) Insect Biochemistry 19, 471-480.
35. Barr, P., Green, K., Gibson, H., Bathurst, I., Quakyi, I. and Kaslow, D. (1991) The Journal of Experimental Medecine, 174, 1203-1208.
36. Coban, C., Philipp, M. T., Purcell, J. E., Keister, D. B., Okulate, M., Martin, D. S. and Kumar, N. (2004) Infect Immun, 72, 253-259.
37. Gozar, M. M., Muratova, O., Keister, D. B., Kensil, C. R., Price, V. L. and Kaslow, D. C. (2001) Exp Parasitol, 97, 61-69.
38. Kaslow, D. C., Bathurst, I. C., Lensen, T., Ponnudurai, T., Barr, P. and Keister, D. B. (1994) Infection and immunity, 62, 5576-5580.
39. Kaslow, D. C., Isaacs, S. N., Quakyi, I. A., Gwadz, R. W., Moss, B. and Keister, D. B. (1991) Science, 252, 1310-1312.
40. Kaslow, D. C., Syin, C., McCutchan, T. F. and Miller, L. H. (1989) Mol Biochem Parasitol, 33, 283-287.
41. Lobo, C. A., Dhar, R. and Kumar, N. (1999) Infection & Immunity, 67, 1688-1693.
42. Vermeulen, A. N., Ponnudurai, T., Beckers, P. G. A., Verhave, J. P., Smits, M. A. and Meuwissen, J. H. E. T. (1985) J. Exp. Med., 162, 1460-1476.
43. Zou, L., Miles, A. P., Wang, J. and Stowers, A. W. (2003) Vaccine, 21, 1650-1657.
44. Almeida, A. P. and Billingsley, P. F. (2002) J Med Entomol, 39, 207-214.
45. Lal, A. A., Schriefer, M. E., Sacci, J. B., Goldman, I. F., Louis-Wileman, V., Collins, W. E. and Azad, A.F. (1994) Infect. immun., 62, N^{o}1, 316-318.
46. Ramasamy, M. S. and Ramasamy, R. (1990) Medical and Veterinary Entomology, 4, 161-166.
47. Srikrishnaraj, A. K., Ramasamy, R. and Ramasamy, M. S. (1995) Medical and Veterinary Entomology, 9, 353-357.
48. Shahabuddin, M., Lemos, F. J. A., Kaslow, D. C. and Jacobslorena, M. (1996) Infection and Immunity, 64, 739-743.
49. Franke-Fayard, B., Trueman, H., Ramesar, J., Mendoza, J., van der Keur, M., van der Linden, R., Sinden, R. E., Waters, A. P. and Janse, C. J. (2004) Mol Biochem Parasitol, 137, 23-33.
50. Vaughan, J. A., Narum, D. and Azad, A. F. (1991) J Parasitol, 77, 758-761.
51. Vaughan, J. A., Noden, B. H. and Beier, J. C. (1992) J Parasitol, 78, 716-724.
52. Han, Y. S., Thompson, J., Kafatos, F. C. and Barillas-Mury, C. (2000) Embo J. 19, 6030-6040.
53. Meis, J. F., Pool, G., van Gemert, G. J., Lensen, A. H., Ponnudurai, T. and Meuwissen, J. H. (1989) Parasitol Res, 76, 13-19.
54. Edwards, M. J., Lemos, F. J., Donnelly-Doman, M. & Jacobs-Lorena, M. (1997) Insect Biochem. Mol. Biol. 27, 1063-72.
55. Ramos, A., Mahowald, A. & Jacobs-Lorena, M. (1993) Insect Mol. Biol. 1, 149-63.
56. Edwards, M. J., Moskalyk, L. A., Donelly-Doman, M., Vlaskova, M., Noriega, F. G., Walker, V. K. & Jacobs-Lorena, M. (2000) Insect Mol Biol 9, 33-8.
57. Clauser, E., Gardell, S., Craik, C., MacDonald, R. and Rutter, W.(1988) J Biol Chem 263: 17837-17845.
58. Guex, N. and Peitsch, M. C. (1997) Electrophoresis 18: 2714-2723.
59. Summers, M. D. and Smith, G. E. (1987) Texas Agric Exp Station Bull, 1555.
60. Moore, S. and Stein, W. H. (1948) J Biol Chem 176:
61. Dardel, F. (1994) Comput Appl Biosci 10: 273-275.
62. Coll, M., Guasch, A., Aviles, F. X. and Huber, R. (1991) Embo J 10:1-9.
63. Dardel, F. (1994) Comput Appl Biosci 10: 273-275.
64. Yamamoto, K., Pousette, A., Chow, P., Wilson, H., el Shami, S. and French, C. (1992) J Biol Chem 267: 2575-2581.
65. Skidgel, R. A. (1996) In Zinc Metalloproteases in Health andDisease (Francis, T. A. E. ed.), pp. 241-309.
66. Titani, K., Ericsson, L. H., Walsh, K. A. and Neurath, H. (1975) Proc Natl Acad Sci USA 72: 1666-1670.
67. Titani, K., Torff, H. J., Hormel, S., Kumar, S., Walsh, K. A., Rodl, J., Neurath, H. and Zwilling, R. (1987) Biochemistry 26: 222-226.
68. Gardell, S., Craik, C., Clauser, E., Goldsmith, E., Stewart, C., Graf, M. and Rutter, W. (1988) J Biol Chem 263: 17828-17836.
69. Tan, A. K. and Eaton, D. L. (1995) Biochemistry 34: 5811-5816.
70. Alter, G. M., Leussing, D. L., Neurath, H. and Vallee, B. L. (1977) Biochemistry 16: 3663-3668.
71. Marinkovic, D. V., Marinkovic, J. N., Erdos, E. G. and Robinson, C. J. (1977) Biochem J 163: 253-260.
72. Bradley, G., Naude, R. J., Muramoto, K., Yamauchi, F. and Oelofsen, W. (1996) Int J Biochem Cell Biol 28: 521-529.
73. Muller, H. M., Catteruccia, F., Vizioli, J., Dellatorre, A. and Crisanti, A. (1995) Exp Parasitol 81: 371-385.
74. Noriega, F. G., Pennington, J. E., Barillas-Mury, C., Wang, X. Y. and Wells, M. A. (1996) Insect Mol Biol 5: 25-29.
75. Graf, R. and Briegel, H. (1989) Insect Biochem 19: 129-137.
76. Barillasmury, C. V., Noriega, F. G. and Wells, M. A. (1995) Insect Biochem Mol Biol 25: 241-246.
77. Noriega, F. G. and Wells, M. A. (1999) J Insect Physiol 45: 613-620.
78. Shen, Z., Edwards, M. J. and Jacobs-Lorena, M. (2000) Insect Mol Biol 9: 223-229.
79. Bown, D. P. and Gatehouse, J. A. (2004) Eur J Biochem 271: 2000-2011.

1. An isolated polynucleotide which encodes a polypeptide comprising an amino acid sequence that is at least 70% homologous to SED ID NO: 2, wherein said polypeptide has carboxypeptidase B activity.

2. The isolated polynucleotide of claim 1, wherein said polynucleotide has a sequence that is at least 70% homologous to nucleotides 32-1300 of SEQ ID NO: 1.

3. The isolated polynucleotide of claim 1, wherein said polynucleotide the sequence of nucleotides 32-1300 of SEQ ID NO: 1.

4. An isolated polynucleotide, which is complementary to the polynucleotide of claim 1.

5. An isolated polynucleotide which hybridizes under stringent conditions to the polynucleotide of claim 1; wherein said stringent conditions comprise washing in 5×SSC at a temperature from 50to 68° C., and wherein said polynucleotide encodes a polypeptide having carboxypeptidase B activity.

6. A vector comprising the isolated polynucleotide of claim 1.

7. The vector of claim 6, wherein the isolated polynucleotide is operably linked to an inducible promoter.

8. A host cell comprising the vector of claim 6.

9. A method for making a polypeptide that is at least 70% homologous to SEQ ID NO: 2 and exhibits carboxypeptidase B activity, comprising culturing the host cell of claim 8 for a time and under conditions suitable for expression of said polypeptide, and collecting the said polypeptide.

10. A process for screening for a polynucleotide which encodes a protein having carboxypeptidase B comprising hybridizing the isolated polynucleotide of claim 4 to the polynucleotide to be screened; expressing the polynucleotide to produce a protein; and detecting the presence or absence of carboxypeptidase B activity in said protein.

11. A method for detecting a polynucleotide having at least 70% homology to the polynucleotide of claim 1, comprising contacting a nucleic acid sample with a probe or primer comprising at least 15 consecutive nucleotides of the polynucleotide of claim 1, or at least 15 consecutive nucleotides of the complement thereof.

12. A method for producing a polynucleotide having at least 70% homology to the polynucleotide of claim 1, comprising:
(a) hybridizing a nucleic acid sample with a primer pair, wherein said primer pair comprises a first primer which is complementary to at least 15 consecutive nucleotides near the 5′end of the polynucleotide of claim 1, and a second primer which is complementary to at least 15 consecutive nucleotides near the 3′ end of the polynucleotide of claim 1, and
(b) synthesizing said polynucleotide.

13. An isolated polypeptide comprising an amino acid sequence, which is at least 70% homologous to SEQ ID NO: 2 and exhibits carboxypeptidase B activity.

14. The isolated polypeptide of claim 13, wherein said polypeptide is at least 70% homologous to amino acid residues 20-423 of SEQ ID NO: 2.

15. The isolated polypeptide of claim 13, wherein said polypeptide is at least 70% homologous to amino acid residues 115-423 of SEQ ID NO: 2.

16. An antibody directed against the polypeptide of claim 13 or an immunogenic fragment thereof.

17. A method of blocking Plasmodium development comprising administering to a subject in need thereof an effective amount of the antibody of claim 16.

18. The method of claim 17, wherein said subject is a mammal.

19. The method of claim 18, wherein said mammal is a human.

20. The method of claim 17, wherein said antibody is a polyclonal antibody.

21. The method of claim 17, wherein said antibody is a monoclonal antibody.

22. A method of blocking plasmodium development comprising administering to a subject in need thereof an effective amount of the polypeptide of claim 13 or an immunogenic fragment thereof.

23. The method of claim 22, wherein said subject is a mammal.

24. The method of claim 23, wherein said mammal is a human.

25. A vaccine comprising the polypeptide of claim 13, or an immunogenic fragment thereof, and at least one pharmaceutically acceptable carrier, adjuvant, diluent, or excipient.

26. A method of identifying compounds that inhibit carboxypeptidase B activity comprising:
contacting the polypeptide of claim 13 or a conformational epitope thereof that possesses carboxypeptidase B activity with a carboxypeptidase B substrate selected from the group consisting of hyppuryl-arginine and hyppuryl-lysine in the presence of a candidate compound, and
measuring the residual carboxypeptidase B activity in the presence of said candidate compound compared to the carboxypeptidase B activity in the absence of said candidate compound.

27. An isolated polynucleotide which encodes a polypeptide comprising an amino acid sequence that is at least 70% homologous to SED ID NO: 4, wherein said polypeptide has carboxypeptidase B activity.

28. The isolated polynucleotide of claim 27, wherein said polynucleotide has a sequence that is at least 70% homologous to nucleotides 76-1341 of SEQ ID NO: 3.

29. The isolated polynucleotide of claim 27, wherein said polynucleotide the sequence of nucleotides 76-1341 of SEQ ID NO: 3.

30. An isolated polynucleotide, which is complementary to the polynucleotide of claim 27.

31. An isolated polynucleotide which hybridizes under stringent conditions to the polynucleotide of claim 27; wherein said stringent conditions comprise washing in 5×SSC at a temperature from 50 to 68° C., and wherein said polynucleotide encodes a polypeptide having carboxypeptidase B activity.

32. A vector comprising the isolated polynucleotide of claim 27.

33. The vector of claim 32, wherein the isolated polynucleotide is operably linked to an inducible promoter.

34. A host cell comprising the vector of claim 27.

35. A method for making a polypeptide that is at least 70% homologous to SEQ ID NO: 2 and exhibits carboxypeptidase B activity, comprising culturing the host cell of claim 34 for a time and under conditions suitable for expression of said polypeptide, and collecting the said polypeptide.

36. A process for screening for a polynucleotide which encodes a protein having carboxypeptidase B comprising hybridizing the isolated polynucleotide of claim 30 to the polynucleotide to be screened; expressing the polynucleotide to produce a protein; and detecting the presence or absence of carboxypeptidase B activity in said protein.

37. A method for detecting a polynucleotide having at least 70% homology to the polynucleotide of claim 27, comprising contacting a nucleic acid sample with a probe or primer comprising at least 15 consecutive nucleotides of the polynucleotide of claim 27, or at least 15 consecutive nucleotides of the complement thereof.

38. A method for producing a polynucleotide having at least 70% homology to the polynucleotide of claim 27, comprising:
(a) hybridizing a nucleic acid sample with a primer pair, wherein said primer pair comprises a first primer which is complementary to at least 15 consecutive nucleotides near the 5′ end of the polynucleotide of claim 27, and a second primer which is complementary to at least 15 consecutive nucleotides near the 3′end of the polynucleotide of claim 27, and
(b) synthesizing said polynucleotide.

39. An isolated polypeptide comprising an amino acid sequence, which is at least 70% homologous to SEQ ID NO: 2 and exhibits carboxypeptidase B activity.

40. The isolated polypeptide of claim 39, wherein said polypeptide is at least 70% homologous to amino acid residues 20-422 of SEQ ID NO: 4.

41. The isolated polypeptide of claim 39, wherein said polypeptide is at least 70% homologous to amino acid residues 108-422 of SEQ ID NO: 4.

42. An antibody directed against the polypeptide of claim 39 or an immunogenic fragment thereof.

43. A method of blocking Plasmodium development comprising administering to a subject in need thereof an effective amount of the antibody of claim 42.

44. The method of claim 43, wherein said subject is a mammal.

45. The method of claim 44, wherein said mammal is a human.

46. The method of claim 43, wherein said antibody is a polyclonal antibody.

47. The method of claim 43, wherein said antibody is a monoclonal antibody.

48. A method of blocking plasmodium development comprising administering to a subject in need thereof an effective amount of the polypeptide of claim 39 or an immunogenic fragment thereof.

49. The method of claim 48, wherein said subject is a mammal.

50. The method of claim 49, wherein said mammal is a human.

51. A vaccine comprising the polypeptide of claim 39, or an immunogenic fragment thereof, and at least one pharmaceutically acceptable carrier, adjuvant, diluent, or excipient.

52. A method of identifying compounds that inhibit carboxypeptidase B activity comprising:
contacting the polypeptide of claim 39 or a conformational epitope thereof that possesses carboxypeptidase B activity with a carboxypeptidase B substrate selected from the group consisting of hyppuryl-arginine and hyppuryl-lysine in the presence of a candidate compound, and
measuring the residual carboxypeptidase B activity in the presence of said candidate compound compared to the carboxypeptidase B activity in the absence of said candidate compound.


SEQUENCE LISTING (66 sequences)

SEQ ID NO: 1 - DNA, 1471 bp, Anopheles gambiae
  CDS (32)..(1300)
agttgtcgtttggcagtggatgagcgaagcaatgaagcggctaacattcgtgaccggctgtttgctggccttggcattcgccaaagccggttcttatcatgaattcgagctgtacaacgtgcgcccggaaacggcggaacagctgtcggtgctgctcaagtggcgcaatgggcaggagattgaggtggacttttgggatgcaccgaaggtgggccgtagcgcacgcctcatggtgaccagggaggatcacaagcgggtggaagagttcctggagcagcacgacatcgagtacgatctggtggcggaggatgtgcaggagttgctgaatcgagagcagcgccgtaatgtggagcacggtcggcggctgaggcgtgactcgaattcgcgtgccactgttaatttcgagcacttctggacgctggacgagatctacgagtatctggacgagctggcggtggcgtacaatgggctggtgcgcgtctcggagatcggtcgtacgcatgaggatcgccccatcaaggccatcacaatctcgaccaggggtgcagtcgatcagacccgtccgattgtgtttatggatggaggtattcatgccagagaatgggccggcgtgatgtcggtcatgtacatgatccacgagtttgtggaacactcggaccagtacgccgagcagctgtccaacacggactacgtcatcgtgccggttgccaacccggacgggtacgtctacacccacgagcagaaccgtctgtggcgcaagaaccgttcgccgggcaatgtactgtgctacggcgtggacctgaaccgcaacttccccttccagtgggatcgtacgacgagtgagtgtacgaacaactttgccggccatgccgcttcctcagaaaacgaaaccaaagcactgatcggactgatggatcagtataaggccgccattcgcatgtacctggcggtgcacacgtacggcgagatgattctgtggccgtggggttacgatttcctgcacgccccgaacgaggacgatctacagcggttgggcgaacgggcacgcgatgcactggtggcggctggcgggcccgagtacgaggtgggcaattcggccgacattctgtacacggcttccggggcgacggacgactacgcgtacagcctgggcgtgccgtactcgtacacgctcgagctgacgggcggtggatcgcaagggttcgatctgcccgcggccgagctggcgcgcgttacctcgcagacgttcgagctgctgaaggtgttcgggcagcatgcgggcacactgtcggtgacttcgtaaagggaagggtgcgtgtgcgtgtgtgtgtgctaatcagccagttgtccgatgtgcgttgattgattatcaattgtatcagcaaagtcgttgctattgagcagtcgcaataaatcaaccattctagcagttacgctatcgcgctctgctttgacattgcaaaaaaaaaaa

SEQ ID NO: 2 - protein, 423 aa, Anopheles gambiae
MKRLTFVTGCLLALAFAKAGSYHEFELYNVRPETAEQLSVLLKWRNGQEIEVDFWDAPKVGRSARLMVTREDHKRVEEFLEQHDIEYDLVAEDVQELLNREQRRNVEHGRRLRRDSNSRATVNFEHFWTLDEIYEYLDELAVAYNGLVRVSEIGRTHEDRPIKAITISTRGAVDQTRPIVFMDGGIHAREWAGVMSVMYMIHEFVEHSDQYAEQLSNTDYVIVPVANPDGYVYTHEQNRLWRKNRSPGNVLCYGVDLNRNFPFQWDRTTSECTNNFAGHAASSENETKALIGLMDQYKAAIRMYLAVHTYGEMILWPWGYDFLHAPNEDDLQRLGERARDALVAAGGPEYEVGNSADILYTASGATDDYAYSLGVPYSYTLELTGGGSQGFDLPAAELARVTSQTFELLKVFGQHAGTLSVTS

SEQ ID NO: 3 - DNA, 1341 bp, Anopheles gambiae
  CDS (76)..(1341)
ccagtcggcattcggtggtcattctgagaaggtgcagtgtcgtgttctgcttatttcgcccaagatacatacagcatgcggtttccagtgctggtggcggttttgtgcgcggtaattggaggtaccgctggccgccaatcgtacagtggctacaagctctactctgtggagcagaacaaccagcagcaggtggacttcctgcgcgagctgcaacagtccgcccaggatcttgacttctggcagcttgatcgtttggttggttccgaggcacgggtgctggtgcctccggcacaattggactacttccgccagctgctgaaggcccagaagttgcgccaccgggaactgattcacgatttcgaacgtgtgcaagatgatcatctgtacggtaccaaacgggtgactccttcgcagggtcttcccctcaacaagtacctgcggtacaacgaaatgatcgactacatcaacacactcgcgaagaagtactccagctttgtaaccgttgccgaaattggcaaatcctacgaaggacgcccaatcccggccgtcaccatccagtccccgtcgctctacaaatcgcacccgaacagctccaaaccggtcgtattcgtcgatgctggcattcatgcgcgtgaatgggccgcacctgccatggcgatgtatctgatcagcgagctggtggagaatgccgcccagcatcaggatctactcgccgggcttacctggacgatcgtgccaattgccaatccggacgggtacgagtacagtcacgagcgggaacgcctgtggcgcaaaacgcgccgtccggccggacgtaactgtgtcggcgtcgatggcaaccgcaactacgacttccactgggcggaggtcggtgcctcgaatcagccctgctccgacacataccacggggagcagtcgttctctgagccggagacgcgcgccatccgggacgagctgctgaggttgaagggacgctgcaagttctacctgtcgctgcacacgtacggacagtacctgctgtacccgtggggatggacgtcggagctgccggaggactggcagaaggtcgatgccgttgcgcgggctggagcacgtgcgattgagcaagcgaccgggtcgacctatacggtcggcagctccaccaatgtgctgtatgcggccgctggaggcagtgatgattacgcgtatgctgtcgccgatgtgccgatttcgatgacgatggaactgccgggcggtggttcgcagggcttcaatccaccgccgacgcgcatcgaagagatcgtgaaggaaacgttcgtgggcgtgcgggcgatggcgttggaggtggcgcgaaactacagc

SEQ ID NO: 4 - protein, 422 aa, Anopheles gambiae
MRFPVLVAVLCAVIGGTAGRQSYSGYKLYSVEQNNQQQVDFLRELQQSAQDLDFWQLDRLVGSEARVLVPPAQLDYFRQLLKAQKLRHRELIHDFERVQDDHLYGTKRVTPSQGLPLNKYLRYNEMIDYINTLAKKYSSFVTVAEIGKSYEGRPIPAVTIQSPSLYKSHPNSSKPVVFVDAGIHAREWAAPAMAMYLISELVENAAQHQDLLAGLTWTIVPIANPDGYEYSHERERLWRKTRRPAGRNCVGVDGNRNYDFHWAEVGASNQPCSDTYHGEQSFSEPETRAIRDELLRLKGRCKFYLSLHTYGQYLLYPWGWTSELPEDWQKVDAVARAGARAIEQATGSTYTVGSSTNVLYAAAGGSDDYAYAVADVPISMTMELPGGGSQGFNPPPTRIEEIVKETFVGVRAMALEVARNYS

SEQ ID NO: 5 - DNA, 18 bp, Artificial Sequence
  Synthetic DNA
ggcggctgaggcgtgact

SEQ ID NO: 6 - DNA, 18 bp, Artificial Sequence
  Synthetic DNA
gacgggtctgatcgactg

SEQ ID NO: 7 - DNA, 20 bp, Artificial Sequence
  Synthetic DNA
caccgccgtgtacgatgcca

SEQ ID NO: 8 - DNA, 20 bp, Artificial Sequence
  Synthetic DNA
atggtggtctgctggttctt

SEQ ID NO: 9 - DNA, 20 bp, Artificial Sequence
  Synthetic DNA
tccggcacaattggactact

SEQ ID NO: 10 - DNA, 20 bp, Artificial Sequence
  Synthetic DNA
taccgcaggtacttgttgag

SEQ ID NO: 11 - DNA, 23 bp, Artificial Sequence
  Synthetic DNA
cgactggagcacgaggacactga

SEQ ID NO: 12 - DNA, 26 bp, Artificial Sequence
  Synthetic DNA
ggacactgacatggactgaaggagta

SEQ ID NO: 13 - DNA, 24 bp, Artificial Sequence
  Synthetic DNA
agcgcgatagcgtaactgctagaa

SEQ ID NO: 14 - DNA, 26 bp, Artificial Sequence
  Synthetic DNA
ttgcgactgctcaatagcaacgactt

SEQ ID NO: 15 - DNA, 42 bp, Artificial Sequence
  Synthetic DNA
ggcgaattccagatctctaagcttcgaagtcaccgacagtgt

SEQ ID NO: 16 - DNA, 41 bp, Artificial Sequence
  Synthetic DNA
taaaagctgaattccgggatcccttcgagctgtacaacgtg

SEQ ID NO: 17 - DNA, 27 bp, Artificial Sequence
  Synthetic DNA
taacggatccggatgagcgaagcaatg

SEQ ID NO: 18 - DNA, 50 bp, Artificial Sequence
  Synthetic DNA
aatctagactattagtggtggtggtggtggtgcgaagtcaccgacagtgt

SEQ ID NO: 19 - DNA, 18 bp, Artificial Sequence
  Synthetic DNA
gggactatctgtgggtgt

SEQ ID NO: 20 - DNA, 19 bp, Artificial Sequence
  Synthetic DNA
gaccgcaatgttcgatccg

SEQ ID NO: 21 - DNA, 19 bp, Artificial Sequence
  Synthetic DNA
acgaaagcaatcggcaaga

SEQ ID NO: 22 - DNA, 19 bp, Artificial Sequence
  Synthetic DNA
ttggacgtgccctggccgc

SEQ ID NO: 23 - DNA, 25 bp, Artificial Sequence
  Synthetic DNA
gccggttgtcttcatcgacggtggt

SEQ ID NO: 24 - DNA, 25 bp, Artificial Sequence
  Synthetic DNA
ttggcaaccggcacgatgacgtagt

SEQ ID NO: 25 - DNA, 25 bp, Artificial Sequence
  Synthetic DNA
ggagaagctgcgcggtaagctcggt

SEQ ID NO: 26 - DNA, 25 bp, Artificial Sequence
  Synthetic DNA
actaaaagcagcgctaagccaccaa

SEQ ID NO: 27 - DNA, 18 bp, Artificial Sequence
  Synthetic DNA
ctactacgcgacgattgc

SEQ ID NO: 28 - DNA, 19 bp, Artificial Sequence
  Synthetic DNA
gttcgggatgatctggtta

SEQ ID NO: 29 - DNA, 18 bp, Artificial Sequence
  Synthetic DNA
tatatcgaatctccaaga

SEQ ID NO: 30 - DNA, 18 bp, Artificial Sequence
  Synthetic DNA
gtgccgtaacgattgtaa

SEQ ID NO: 31 - DNA, 26 bp, Artificial Sequence
  Synthetic DNA
ggtcgggaaggaaacgctcgcggcat

SEQ ID NO: 32 - DNA, 26 bp, Artificial Sequence
  Synthetic DNA
actccactctcacgcacagggggaac

SEQ ID NO: 33 - DNA, 19 bp, Artificial Sequence
  Synthetic DNA
taagtatcagtggatcgtt

SEQ ID NO: 34 - DNA, 19 bp, Artificial Sequence
  Synthetic DNA
tattgcgcgactttctggc

SEQ ID NO: 35 - DNA, 18 bp, Artificial Sequence
  Synthetic DNA
ctctgggccatcgccatt

SEQ ID NO: 36 - DNA, 19 bp, Artificial Sequence
  Synthetic DNA
ggtcagcggagtttgaaga

SEQ ID NO: 37 - DNA, 18 bp, Artificial Sequence
  Synthetic DNA
ttccactctcacgtaagc

SEQ ID NO: 38 - DNA, 18 bp, Artificial Sequence
  Synthetic DNA
caccccgtacggttgcat

SEQ ID NO: 39 - DNA, 25 bp, Artificial Sequence
  Synthetic DNA
atcaataggcagcaagagaagaaaa

SEQ ID NO: 40 - DNA, 25 bp, Artificial Sequence
  Synthetic DNA
ggatttttatcgtcggggtttacct

SEQ ID NO: 41 - DNA, 25 bp, Artificial Sequence
  Synthetic DNA
ttcatatttctagactatcccacac

SEQ ID NO: 42 - DNA, 24 bp, Artificial Sequence
  Synthetic DNA
gctctggacggtaagaatggagga

SEQ ID NO: 43 - DNA, 25 bp, Artificial Sequence
  Synthetic DNA
cgccgcaccaaaacacgctacctaa

SEQ ID NO: 44 - DNA, 25 bp, Artificial Sequence
  Synthetic DNA
accttcgtgtaggtgttgcgcttca

SEQ ID NO: 45 - DNA, 18 bp, Artificial Sequence
  Synthetic DNA
acaaccgataaggcacca

SEQ ID NO: 46 - DNA, 18 bp, Artificial Sequence
  Synthetic DNA
cactgccattgctgtaat

SEQ ID NO: 47 - DNA, 18 bp, Artificial Sequence
  Synthetic DNA
ggactataagtgattatc

SEQ ID NO: 48 - DNA, 18 bp, Artificial Sequence
  Synthetic DNA
gtgcaagatccgatagtg

SEQ ID NO: 49 - DNA, 26 bp, Artificial Sequence
  Synthetic DNA
ttcgcaagctgcggcctgcgtgcaag

SEQ ID NO: 50 - DNA, 25 bp, Artificial Sequence
  Synthetic DNA
gcgccaccggtaccttctcgatgtc

SEQ ID NO: 51 - DNA, 24 bp, Artificial Sequence
  Synthetic DNA
gtggaacaccgttggtggaagcaa

SEQ ID NO: 52 - DNA, 24 bp, Artificial Sequence
  Synthetic DNA
aacgctgttggcaagattgcgcag

SEQ ID NO: 53 - DNA, 25 bp, Artificial Sequence
  Synthetic DNA
caagcattcgaagaactcaaacaca

SEQ ID NO: 54 - DNA, 25 bp, Artificial Sequence
  Synthetic DNA
ctggattcgctatcggaataatgat

SEQ ID NO: 55 - DNA, 25 bp, Artificial Sequence
  Synthetic DNA
cggccaaacttcgcctacaatcaga

SEQ ID NO: 56 - DNA, 25 bp, Artificial Sequence
  Synthetic DNA
ccaaaggtacgtatctcgtatgacc

SEQ ID NO: 57 - DNA, 18 bp, Artificial Sequence
  Synthetic DNA
atcgtatgaagatactca

SEQ ID NO: 58 - DNA, 18 bp, Artificial Sequence
  Synthetic DNA
tatacccttcccgcagga

SEQ ID NO: 59 - DNA, 26 bp, Artificial Sequence
  Synthetic DNA
ggtagaggagaacgtacagtcactgc

SEQ ID NO: 60 - DNA, 24 bp, Artificial Sequence
  Synthetic DNA
atcgatcgcatcctcatcgacttc

SEQ ID NO: 61 - protein, 417 aa, Homo sapiens
MLALLVLVTVALASAHHGGEHFEGEKVFRVNVEDENHINIIRELASTTQIDFWKPDSVTQIKPHSTVDFRVKAEDTVTVENVLKQNELQYKVLISNLRNVVEAQFDSRVRATGHSYEKYNKWETIEAWTQQVATENPALISRSVIGTTFEGRAIYLLKVGKAGQNKPAIFMDCGFHAREWISPAFCQWFVREAVRTYGREIQVTELLDKLDFYVLPVLNIDGYIYTWTKSRFWRKTRSTHTGSSCIGTDPNRNFDAGWCEIGASRNPCDETYCGPAAESEKETKALADFIRNKLSSIKAYLTIHSYSQMMIYPYSYAYKLGENNAELNALAKATVKELASLHGTKYTYGPGATTIYPAAGGSDDWAYDQGIRYSFTFELRDTGRYGFLLPESQIRATCEETFLAIKYVASYVLEHLY

SEQ ID NO: 62 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

SEQ ID NO: 63 - protein, 401 aa, Sus scrofa
HHSGEHFEGEKVFRVNVEDENDISELHELASTRQIDFWKPDSVTQIKPHSTVDFRVKAEDILAVEDFLEQNELQYEVLINNLRSVLEAQFDSRCRTTGHSYEKYNNWETIEAWTEQVTSKNPDLISRSAIGTTFDGDNIYLLKVGKPGSNKPAIFMDCGFHAREWISQAFCQWFVRDAVRTYGYEAHMTEFLDNLDFYVLPVLNIDGYIYTWTKNRMWRKTRSTNAGSSCTGTDPNRNFNAGWCTVGASVNPCNETYCGSAAESEKETKALADFIRNNLSSIKAYLTIHSYSQMILYPYSYDYKLPENDAELNSLAKGAVKELASLYGTSYSYGPGSTTIYPAAGGSDDWAYNQGIKYSFTFELRDKGRFGFVLPESQIQATCQETMLAVKYVTNYTLEHL

SEQ ID NO: 64 - protein, 415 aa, Rattus norvegicus
MLLLLALVSVALAHASEEHFDGNRVYRVSVHGEDHVNLIQELANTKEIDFWKPDSATQVKPLTTVDFHVKAEDVADVENFLEENEVHYEVLISNVRNALESQFDSHTRASGHSYTKYNKWETIEAWIQQVATDNPDLVTQSVIGTTFEGRNMYVLKIGKTRPNKPAIFIDCGFHAREWISPAFCQWFVREAVRTYNQEIHMKQLLDELDFYVLPVVNIDGYVYTWTKDRMWRKTRSTMAGSSCLGVRPNRNFNAGWCEVGASRSPCSETYCGPAPESEKETKALADFIRNNLSTIKAYLTIHSYSQMMLYPYSYDYKLPENYEELNALVKGAAKELATLHGTKYTYGPGATTIYPAAGGSDDWSYDQGIKYSFTFELRDTGFFGFLLPESQIRQTCEETMLAVKYIANYVREHLY

SEQ ID NO: 65 - protein, 433 aa, Anopheles gambiae
MVRLNSAVGSRWWAPAMAILAVALSVEAAEVARYDNYRLYRVTPHSEAQLRSVAAMEQASDSLIFLETARKLGDRFDIVVAPHKLADFTETLESDYIPHELIEQNVQRAFDEERVRLTNKRAKGPFDWNDYHTLEEIHAWLDQLASEHPKEVELLDAGRSHQNRTMKGVKLSYGPGRPGVFLEGGIHAREWISPATVTYILNQLLTSEDAKVRALAEKFDWYVFPNANPDGYAYTFQVNRLWRKTRKAYGPFCYGADPNRNWDFHWAEQGTSNNACSDTYHGSEAFSEVETRSLAAFVEKLRGKLGAYIAFHSYSQLLLFPYGDTGAHCGNHQDLNEIAEATVKSLAKRYGTQYKYGNVIDAIYPASGSSVDWSYGAQDVKIAYTYELRPDGDAWNGFVLPPNEIVPTGEETLDSLITLLEESSARGYYDEKH

SEQ ID NO: 66 - protein, 427 aa, Aedes aegypti
MGLPVAVTVLGICLCFGVTYGNEAARYDNYRVYEAIPSSNSQLELLNELEQSSDSLIFLKSGNNVGEKFNIVVAPHKLVDFTEALQNEGVRVRLLETNMQSLIDEEKQRMVSKRARGAFDFNDYYELEDIHAWLDKLANQYDQVQLLEGGHSFENRSIKGVKVSYKTGNPGIFVEGGIHAREWISPATVAYILNELLTSTDPKVRNIAENYDWYMFPSVNPDGYVYTHKKDRLWRKTRTPYSGGCFGADPNRNWDFHWAEQGTSNRCNSDTYGGPHAFSEVETKSLSQFIASLKGKIQAYISFHSYSQLLLFPYGHTGEHASNHNDLNEIAKATITSLAKRYGTKYKYGNIYDAIYPASGASVDWAYGTLDVKIAYTYELRPGSGSWNGFVLPPKQIVPTGEETLDSLVTLLEESDKRGYYENCEGC